(19) United States
(12) Patent Application Publication
(10) Pub. No.: US 2022/0372451 A1
(43) Pub. Date: Nov. 24, 2022

(54) PRODUCTION OF PYRUVATE OR PRODUCTS DERIVED FROM PYRUVATE USING MODIFIED ESCHERICHIA COLI

(71) Applicant: University of Georgia Research Foundation, Inc., Athens, GA (US)

(72) Inventors: Mark Andrew Eiteman, Athens, GA (US); William Chris Moxley, Athens, GA (US)

(21) Appl. No.: 17/729,120

(22) Filed: Apr. 26, 2022

Related U.S. Application Data
(60) Provisional application No. 63/180,719, filed on Apr. 28, 2021.

(51) Int. Cl. C12N 9/02; C12P 7/24

(52) U.S. Cl. CPC C12N 9/0008; C12P 7/24

(57) ABSTRACT

Modified bacterial strains are provided. The strains can generate a desired product such as pyruvate and products derived from pyruvate. Methods of generating pyruvate and products derived from pyruvate are also provided. The modified bacterial strains have at least one mutation in a gene coding for proteins in a pyruvate dehydrogenase complex such that the mutation allows a cell to accumulate pyruvate and/or products derived from pyruvate.

### CROSS-REFERENCE TO RELATED APPLICATIONS

This application claims the benefit of and priority to U.S. Provisional Application Ser. No. 63/180,719, having the title “PRODUCTION OF PYRUVATE OR PRODUCTS DERIVED FROM PYRUVATE USING MODIFIED ESCHERICHIA COLI”, filed on Apr. 28, 2021, the disclosure of which is incorporated herein by reference in its entirety.

### STATEMENT REGARDING FEDERALLY SPONSORED RESEARCH OR DEVELOPMENT

This invention was made with Government support under grant number 1802533 awarded by the National Science Foundation and under grant number 2014-38502-22598 awarded by the US Department of Agriculture, National Institute of Food and Agriculture. The Government has certain rights in the invention.

### SEQUENCE LISTING

The instant application contains a sequence listing which has been submitted with the instant application via EFS-Web. The sequence listing file named 222105-1100_ST25.txt created on May 20, 2022 is 12 kilobytes in size and is incorporated herein by reference in its entirety.

### BACKGROUND

Microbial production of biochemicals from renewable resources has become an efficient and cost-effective alternative to traditional chemical synthesis methods. Metabolic engineering tools are important for optimizing a process to perform at an economically feasible level. Additional tools are needed to modify central metabolism and direct metabolic flux to a desired product, such as pyruvate. These needs and other needs are satisfied by the present disclosure.

### SUMMARY

Embodiments of the present disclosure provide modified bacterial strains that can generate a desired product and methods of generating said product, and methods of generating pyruvate and products derived from pyruvate.

An embodiment of the present disclosure includes a modified bacterial strain comprising at least one mutation in a gene coding for proteins in a pyruvate dehydrogenase complex, wherein the mutation allows a cell to accumulate a product.

An embodiment of the present disclosure includes a modified bacterial strain comprising at least one mutation in a gene coding for proteins in a pyruvate dehydrogenase complex, wherein the at least one mutation allows a cell to accumulate pyruvate.

An embodiment of the present disclosure also includes a modified bacterial strain that includes at least one mutation in a gene coding for proteins in a pyruvate dehydrogenase complex. The at least one mutation allows a cell to accumulate acetoin.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in

FIG. 1 provides a comparison of E. coli IdhA poxB AceE variants grown in shake flasks with 5 g/L glucose: specific growth rate (h⁻¹, gray bars) and pyruvate yield (g/g, black bars). WT AceE indicates wild-type AceE. Error bars indicate standard deviation from three replicates.

FIGS. 2A-2D show controlled 1.0 liter batch growth of E. coli AceE variants with 15 g/L glucose. FIG. 2A) MEC825 (C IdhA poxB aceE::aceE); FIG. 2B) MEC961 (C IdhA poxB aceE::aceE ppsA); FIG. 2C) MEC826 (C IdhA poxB aceE::aceE^{[H106V]}); FIG. 2D) MEC905 (C IdhA poxB aceE::aceE^{[H106V]} ppsA). Glucose (▪), pyruvate (▴), OD (◯).

FIG. 3 provides comparison of glucose uptake rate (g/gh, ▴, Δ) and pyruvate yield (g/g, •, ◯) of MEC905 (C IdhA poxB aceE::aceE^{[H106V]} ppsA, filled symbols) and MEC961(C IdhA poxB aceE::aceE ppsA, unfilled symbols) during steady-state growth at the indicated dilution rates (h⁻¹).

FIGS. 4A-4B show growth and pyruvate formation in a controlled 1.0 liter repeated-batch process with 15 g/L glucose and 2 g/L acetate. A 17 mL solution containing 15 g of glucose (and no acetate) was added when the initial glucose was depleted. FIG. 4A) MEC992 (C IdhA poxB aceE ppsA); FIG. 4B) MEC994 (C IdhA poxB aceE::aceE^{[H106M;E401A]} ppsA). Glucose (▪), pyruvate (▴), acetate (•), OD (◯).

FIG. 5 shows biochemical pathways for the conversion of glucose to acetoin production in E. coli. Gene knockouts performed in this study are indicated by solid red “x”. Flux through the PDHc was partly curtailed (dotted red “x”) by the introduction of aceE variant alleles coding amino acid substitutions in the E1 component of the complex (X, solid, red). Heterologous budB and budA genes from Enterobacter cloacae ssp. dissolvens were expressed for the conversion of pyruvate to acetoin (green).

FIG. 6 provides a comparison of E. coli W IdhA poxB ppsA AceE variants grown in shake flasks with 5 g/L glucose: specific growth rate (h⁻¹, gray bars) and pyruvate yield (g/g, black bars). Error bars indicate standard deviation from three replicates.

FIG. 7 provides a comparison of E. coli W IdhA poxB ppsA AceE variants harboring 44_ediss grown in shake flasks with 5 g/L glucose: acetoin yield (g/g, grey bars). Error bars indicate standard deviation from three replicates. MEC1342/44_ediss and MEC1322/44_ediss were supplemented with 1 g/L acetate to support growth.

FIGS. 8A-8D graph controlled 1.25 liter batch growth of E. coli AceE variants harboring 44_ediss with 40 g/L glucose. Glucose (♦), pyruvate (▾), OD (●), acetate (▴), acetoin (▪). FIG. 8A: MEC1319/44_ediss (W IdhA poxB ppsA); FIG. 8B: MEC1340 (W IdhA poxB ppsA aceE::aceE^{[V169A;P190Q;F532L]}); FIG. 8C: MEC1332/44_ediss (W IdhA poxB ppsA aceE::aceE^{[H106V]}); FIG. 8D: MEC1342/44_ediss (W IdhA poxB ppsA aceE::aceE^{[H106M;E401A]}).

FIG. 9 shows graphs controlled 1.25 liter repeated-batch growth of MEC1332/44_ediss (W IdhA poxB ppsA aceE::aceE^{[H106V]}) with 40 g/L glucose. 35 g glucose was added to the medium three times when glucose depleted. Glucose (♦), pyruvate (▾), OD (●), acetoin (▪).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of microbiology which are within the skill of the art.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### General Discussion

In accordance with the purpose(s) of the present disclosure, as embodied and broadly described herein, embodiments of the present disclosure, in some aspects, relate to modified bacterial strains that can generate a desired product and methods of generating said product.

In general, embodiments of the present disclosure provide for methods of generating pyruvate and products derived from pyruvate.

The present disclosure includes a modified bacterial strain having one or more mutations that allows a cell to accumulate a desired product. The mutation(s) can be in a gene coding for proteins in the pyruvate dehydrogenase complex. Advantageously, the present methods and strains result in product accumulation while reducing or preventing conversion of the desired product to other products. Additionally, the supplementation of an additional carbon source is not required. The mutation(s) results in reduced activity of the pyruvate dehydrogenase complex and the desired product is accumulated from glucose during aerobic growth.

In some embodiments, the desired product can be pyruvate or products derived from pyruvate. In some embodiments, the product derived from pyruvate can be acetoin. Other desired products can include but are not limited to 2,3-butanediol, diacetyl, L-valine, L-alanine, isobutanol, isoleucine, 2-ketoisovaleric acid.

In some embodiments, the modified bacterial strain is Escherichia coli. Advantageously, unlike other strains that are native acetoin and 2,3-butanediol producers, E. coli is generally regarded as safe. In some embodiments, the E. coli strain can be E. coli W, E. coli C, E. coli B, etc. In other embodiments, the modified bacterial strain can be such as Klebsiella oxytoca, Klebsiella pneumoniae, Lactococcus lactis, Bacillus licheniformis, Bacillus pumilus, Bacillus subtilis, Bacillus amyloliquefaciens, Shewanella oneidensis, Serratia marcescens, Paenibacillus polymyxa, Enterobacter cloacae, Enterobacter aerogenes, Corynebacterium glutamicum, or other strains as can be envisioned by one of ordinary skill in the art.

In some embodiments, the mutated gene is aceE. The site of the mutation can be at one or more of amino acid residues [H106], [V169], [P190], [N276], [E401], [R465], [F532], [V668], and [Y696]. In particular embodiments, the mutation can be at aceE[E401A], aceE[H106V], or aceE[H106M]. For reference, the present disclosure denotes a gene in the format “aceE” and a protein in the format “AceE”. In a particular embodiment, a first point mutation can be at [H106] and a second can be at [E401]. For example, the first mutation could be at aceE[H106V] or aceE[H106M] and the second could be at aceE[E401A]. In some embodiments, the modified strain is not E. coli but may have sequences that are the same length as E. coli. In such cases (e.g. Klebsiella oxytoca, Klebsiella pneumoniae and Enterobacter cloacae), the modified strain can have amino acid residues in alignment with E. coli, such that the proteins occupy the same location in the non-E. coli strain as in E. coli. In non-limiting examples, H106 exists in e.g. Klebsiella oxytoca, Klebsiella pneumoniae and Enterobacter cloacae in the corresponding location in E. coli.

In other embodiments, the modified strain can produce pyruvate or products derived from pyruvate and can have a different sequence length and/or alignment from E. coli. For example, these strains may not have an exact analog (e.g. an H106 analog), or it may be located in a different position (e.g. H100 or H103). It can be appreciated by one of ordinary skill in the art that proteins from different microorganisms may have slightly different positions in the linear sequence. This disclosure is intended to encompass proteins that would be recognized as a pyruvate dehydrogenase protein in a given microorganism and display the same and/or similar activity as the pyruvate dehydrogenase protein (e.g. producing a residue corresponding to the H106 residue on the E. coli AceE sequence, a residue corresponding to the V169 residue on the E. coli AceE sequence, a residue corresponding to the P190 residue on the E. coli AceE sequence, etc.).

In some embodiments, the mutated gene is aceE. The site of the mutation can be at one or more of amino acid residues that affect the binding of cofactors, coenzymes and substrates, such as thiamine pyrophosphate and pyruvate, and/or destabilize the native protein structure.

In some embodiments, the mutation can be in an E1 component of the pyruvate dehydrogenase complex, an E2 component of the pyruvate dehydrogenase complex, or an E3 component of the pyruvate dehydrogenase complex.

In some embodiments, the mutation(s) can include a deletion of phosphoenolpyruvate synthase (ppsA). In other embodiments, the mutations can include a deletion of lactate dehydrogenase (IdhA) and pyruvate oxidase (poxB).

Embodiments of the present disclosure include a modified bacterial strain as above, wherein the mutation(s) in the gene coding for proteins are in the pyruvate dehydrogenase complex and the mutation(s) allow the cell to accumulate pyruvate.

In some embodiments, the mutation(s) result in reduced activity of the pyruvate dehydrogenase complex and pyruvate is accumulated from glucose during aerobic growth.

In some embodiments, the strain yields about 0.50 g to 1.0 g of pyruvate per gram of glucose, about 0.50 to 0.80 g of pyruvate per gram of glucose, or about 0.6 to 0.75 g of pyruvate per gram of glucose.

In some embodiments, the strain generates about 1.00 g/Lh to 2.00 g/Lh of pyruvate or about 0.90 g/Lh of pyruvate.

Embodiments of the present disclosure include a modified bacterial strain as above, wherein the mutation(s) in the gene coding for proteins are in the pyruvate dehydrogenase complex and the mutation(s) allow the cell to accumulate acetoin.

In some embodiments, the mutation results in reduced activity of the pyruvate dehydrogenase complex, and acetoin is accumulated from glucose during aerobic growth.

In some embodiments, the strain yields about 0.20 g to 0.30 g of acetoin per gram of glucose or up to about 0.48 g of acetoin per gram of glucose.

In some embodiments, the strain generates at least 0.80 g/Lh of acetoin. In some embodiments, the strain generates up to 1.06 g/Lh of acetoin.

Embodiments of the present disclosure also provide for methods of producing pyruvate and products derived from pyruvate. The methods can include preparing a modified bacterial strain as provided above. The strain can have at least one mutation in the pyruvate dehydrogenase complex such that activity of the pyruvate dehydrogenase complex is reduced and a product (e.g. pyruvate and/or products derived from pyruvate) is accumulated from glucose during aerobic growth.

### EXAMPLES

### Example 1

Altering metabolic flux at a key branchpoint in metabolism has commonly been accomplished through gene knockouts or by modulating gene expression. An alternative approach to direct metabolic flux preferentially toward a product is decreasing the activity of a key enzyme through protein engineering. In Escherichia coli, pyruvate can accumulate from glucose when carbon flux through the pyruvate dehydrogenase complex is suppressed. Based on this principle, 16 chromosomally expressed AceE variants were constructed in E. coli C and compared for growth rate and pyruvate accumulation using glucose as the sole carbon source. To prevent conversion of pyruvate to other products, the strains also contained deletions in two nonessential pathways: lactate dehydrogenase (IdhA) and pyruvate oxidase (poxB). The effect of deleting phosphoenolpyruvate synthase (ppsA) on pyruvate assimilation was also examined. The best pyruvate-accumulating strains were examined in controlled batch and continuous processes. In a nitrogen-limited chemostat process at steady-state growth rates of 0.15-0.28 h⁻¹, an engineered strain expressing the AceE[H106V] variant accumulated pyruvate at a yield of 0.59-0.66 g pyruvate/g glucose with a specific productivity of 0.78-0.92 g pyruvate/g cells·h⁻¹. These results provide proof-of-concept that pyruvate dehydrogenase complex variants can effectively shift carbon flux away from central carbon metabolism to allow pyruvate accumulation. This approach can potentially be applied to other key enzymes in metabolism to direct carbon toward a biochemical product.

Microbial production of biochemicals from renewable resources has become an efficient and cost-effective alternative to traditional chemical synthesis methods. Metabolic engineering tools are important for optimizing a process to perform at an economically feasible level. The present disclosure describes an additional tool to modify central metabolism and direct metabolic flux to a product. We have shown that variants of the pyruvate dehydrogenase complex can direct metabolic flux away from cell growth to increase pyruvate production in Escherichia coli. This approach could be paired with existing strategies to optimize metabolism and create industrially relevant and economically feasible processes.

### Introduction

A well-optimized microorganism and process are important for microbial conversion of inexpensive substrates like glucose to biochemicals at high yields and productivities. Directing metabolic flux from central metabolism to a biochemical product inherently involves a competition between native metabolism (i.e., cell growth) and the typically synthetic pathway toward that product. Commonly, the focus for improving the process lies in the pathway from central metabolism leading to that biochemical (the “product pathway”), for example, by increasing the activity of enzymes in that pathway (1) or by elevating the expression of the introduced genes (2, 3).

One common approach to reduce the competition between the product pathway and native enzymes is to knockout genes of nonessential pathways that compete for precursor metabolites. For example, Escherichia coli with deletions in genes to native fermentation and acetate assimilation pathways allow acetate to accumulate as the main fermentation product (4). Similarly, E. coli with an inactive phosphoenolpyruvate (PEP)-consuming glucose phosphotransferase system directs more PEP toward the aromatic pathway due to increased PEP availability (5, 6). In some cases, gene knockouts result in auxotrophy, necessitating medium supplementation. For example, L-glutamate and L-leucine are required for growth of citramalate-producing E. coli strains having deletions in leuCD and gitA (7). Similarly, shikimate formation using E. coli aroK aroL creates an aromatic amino acid requirement (8).

A gene knockout typically represents the complete elimination of a flux. Modulating the activity of an enzyme is potentially more beneficial than completely shutting off a pathway. One approach to modulating flux is to use strains auxotrophic for specific enzyme cofactors or affectors, which then are limited in the medium to control flux through a pathway. For example, thiamine or lipoic acid auxotrophs can accumulate pyruvate (9-11). The auxotroph grown in a thiamine- or lipoic acid-limiting medium reduces the specific activity of the pyruvate dehydrogenase complex (PDHc), leading to increased pyruvate accumulation. Similarly, manganese limitation increases citric acid accumulation by Aspergillus niger (12, 13).

Another sophisticated method for controlling the enzyme activity during a process is through promoter engineering, which involves either a static or dynamic control of the expression level of key enzymes. The most common approach for promoter engineering is increasing the expression of enzymes in the product pathway (1, 14, 15). Alternatively, the expression of a competing enzyme in native metabolism can be diminished by introducing a weaker promoter. For example, Corynebacterium glutamicum produces more L-valine by down-modulating a gene in a competing pathway (ilvA) while up-modulating two genes in the L-valine synthesis pathway (ilvD and ilvE) (16). Promoter engineering affects specific enzyme activity by decreasing the concentration of the native enzyme and does not alter the kinetic parameters of that enzyme (i.e., k_{cat} and Kₘ). Promoter engineering can have unpredictable effects on existing metabolic networks (17) and may require the maintenance of plasmid DNA (18, 19).

The central metabolite pyruvate is produced by microbial processes (20) and is also a metabolic precursor for several biochemicals such as isobutanol and 2,3-butanediol (21). Metabolic engineering strategies used to accumulate pyruvate commonly include inactivating pyruvate-consuming pathways. In E. coli, for example, by-product pathways that convert pyruvate to acetate and lactate are blocked respectively by inactivating poxB and IdhA (22, 23). Under aerobic conditions, the majority of pyruvate is converted into acetyl CoA by the PDHc (24), therefore controlling metabolic flux toward acetyl CoA is important for accumulation of pyruvate and products derived from pyruvate.

The PDHc is a large multi-unit complex of three different enzymes. The first dehydrogenase, the E1 component (or AceE) coded by aceE, converts pyruvate to CO₂ and transfers the remaining hydroxyethyl group onto an enzyme-bound thiamine diphosphate (ThDP) and subsequently to a lipoate moiety of the adjacent E2 component coded by aceF. The E2 component transfers acetyl to CoA forming acetyl CoA, while the remaining dihydrolipoate is reoxidized by the E3 component coded by IpdA forming NADH. One strategy to accumulate pyruvate is by the deletion of any one of these three PDHc enzyme components (22, 23). However, PDHc-deficient E. coli strains cannot synthesize acetyl CoA from pyruvate under aerobic conditions and require a secondary carbon source such as acetate (25). Additional nutrient requirements increase operating costs of the process; therefore, an appealing alternative is instead to decrease the activity of the complex. In addition to thiamine or lipoic acid auxotrophy to control activity of the complex, another strategy for pyruvate accumulation is oxygen limitation to encourage elevated concentrations of NADH, which inhibits the E3 component (26, 27). Promoter engineering can also decrease the PDHc activity by decreasing the expression of one of the subunits of the complex. For example, the native aceE promoter in Corynebacterium glutamicum was replaced by weaker promoter variants to produce a range of growth rates and PDHc activities (28). Subsequent overexpression of L-valine biosynthetic genes ilvBNCE resulted in all variants accumulating more L-valine than the strain with the native promoter.

An alternate approach to direct metabolic flux preferentially toward a product pathway is to decrease the intrinsic activity of the competing native enzyme at that metabolic branchpoint. Changing the intrinsic activity could be accomplished by altering key residues which affect substrate binding (i.e., Kₘ) or turnover (i.e., k_{cat}). Alteration of substrate affinity in a highly active native enzyme would allow the product pathway to be more competitive. This approach could potentially be combined with promoter engineering or other methods to allow greater flexibility to optimize carbon flux to a desired product. Since the E1 component of PDHc is the rate-limiting step, the AceE protein is an appropriate target for reducing flux between pyruvate and acetyl CoA.

The goal of the study provided herein was to create variants of PDHc which reduce the native carbon flux to acetyl CoA and shift flux to pyruvate. We hypothesized that PDHc variants of E. coli having reduced activity would accumulate pyruvate from glucose during aerobic growth. In order to prevent conversion of pyruvate to other products, the strains also contained deletions in two nonessential pathways: lactate dehydrogenase (IdhA) and pyruvate oxidase (poxB).

### Material and Methods

Strains and Genetic Modifications

Strains used in this study are shown in Table 1. The ppsA gene knockout in E. coli C (ATCC 8739) was constructed by methods previously described (29). The aceE, IdhA, and poxB gene knockout strains were acquired from the Keio (FRT)Kan collection (30). Gene knockouts were transduced into recipient strains by P1 phage transduction. Knockouts were selected on Lysogeny Broth (LB) orTYA (31) plates supplemented with kanamycin. Forward primers external to the target gene and reverse primers within the kanamycin resistance cassette were used to confirm proper chromosomal integration. The kan^{R} marker was removed by expression of FLP recombinase from pCP20 (29). Gene knockouts and removal of the markers were verified by PCR. To construct MEC813, a chloramphenicol-sacB (cam-sacB) cassette and 500 bp of homology flanking aceE was amplified from pCM03 and integrated into the aceE locus of MEC785 expressing the lambda red system from pKD46. Flanking regions surrounding aceE were sequence verified.

A scarless approach was used to integrate point-mutated aceE variants (32). pKSI-1 harboring a point-mutated aceE was used as donor DNA. If integration was unsuccessful using the method above, the point-mutated aceE variant and 500 bp of flanking homology were amplified from the respective plasmid and used to transform electrocompetent MEC813 expressing the lambda red system from pKD46 (29). Counter-selection against sacB was used to select mutants that lost the cam-sacB cassette by plating transformants on medium containing sucrose (33). Colonies were confirmed by colony PCR, and point-mutated aceE genes were amplified from the chromosome, gel purified, and sequenced to confirm mutations.

TABLE 1
Strains used in this study*.

| Strain | Relevant characteristics | Reference |
|---|---|---|
| ATCC | Escherichia coli C | Wild-type |
| 8739 | | |
| MEC785 | ATCC 8739 aceE732::(FRT) ldhA744::(FRT) | This study |
| | poxB772::(FRT) | |
| MEC813 | MEC785 aceE::cam-sacB 869, 658-872, 250 | This study |
| | (flanked by I-SceI sites) | |
| MEC817 | MEC813 aceE::aceE^{[H106N]} | This study |
| MEC825 | MEC813 aceE::aceE | This study |
| MEC826 | MEC813 aceE::aceE^{[H106V]} | This study |
| MEC827 | MEC813 aceE::aceE^{[E401A]} | This study |
| MEC860 | MEC813 aceE::aceE^{[H106M]} | This study |
| MEC861 | MEC813 aceE::aceE^{[E401D]} | This study |
| MEC862 | MEC813 aceE::aceE^{[K403A]} | This study |
| MEC863 | MEC813 aceE::aceE^{[K403N]} | This study |
| MEC864 | MEC813 aceE::aceE^{[K403Q]} | This study |
| MEC865 | MEC813 aceE::aceE^{[K410M]} | This study |
| MEC866 | MEC813 aceE::aceE^{[K410N]} | This study |
| MEC867 | MEC813 aceE::aceE^{[K410Q]} | This study |
| MEC868 | MEC813 aceE::aceE^{[G395A]} | This study |
| MEC905 | MEC826 ppsA::(FRT) | This study |
| MEC918 | MEC813 aceE::aceE^{[H142V]} | This study |
| MEC919 | MEC813 aceE::aceE^{[H640V]} | This study |
| MEC955 | MEC813 aceE::aceE^{[Y11F]} | This study |
| MEC956 | MEC813 aceE::aceE^{[H106M; E401A]} | This study |
| MEC961 | MEC825 ppsA::(FRT) | This study |
| MEC992 | MEC785 ppsA::(FRT) | This study |
| MEC994 | MEC956 ppsA::(FRT) | This study |

*double colon indicates a replacement of an allele.

Plasmid Construction

Plasmids used in this study are listed in Table 2, and primers used in this study are listed in Table 3. pKSI-1 (Addgene plasmid #51725; http://n2t.net/addgene:51725; RRID:Addgene_51725) and pREDTKI (Addgene plasmid #51628; http://n2t.net/addgene:51628 RRID:Addgene_51628) were gifts from Sheng Yang (32). Plasmids were constructed using NEBuilder HiFi Assembly (New England Biolabs, pswich, MA, USA) or Escherichia coli DH5α-mediated assembly (35). Phusion High-Fidelity Polymerase (New England Biolabs, Ipswich, Mass., USA) or PrimeStar Max High-Fidelity Polymerase (Takara Bio, Mountain View, Calif., USA) was used to amplify DNA for cloning and genome integration. Quick-DNA Miniprep and Zyppy Plasmid Miniprep Kits were used to purify genomic and plasmid DNA (Zymo Research, Irvine, Calif., USA). DNA Clean and Concentrator and Zymoclean Gel DNA Recovery Kits were used to purify PCR fragments (Zymo Research, Irvine, Calif., USA). Restriction enzymes were purchased from New England Biolabs. Plasmids were confirmed by restriction digest and sequencing (ACGT, Inc., Wheeling, Ill., USA).

TABLE 2
Plasmids used in this study.

| | Relevant | | |
|---|---|---|---|
| Name | characteristics | Description | Source |
| pKD4 | Amp^{R}, Kan^{R}; R6K ori | Source of Kan^{R} | 29 |
| | | cassette | |
| pKD46 | Amp^{R}; pSC101 ori | λ Red helper plasmid | 29 |
| | (ts); araBAD promoter | | |
| | for λ Red genes | | |
| pCP20 | Amp^{R}, Cam^{R}; pSC101 | Expression of FLP | 29 |
| | ori (ts) | recombinase | |
| pEL04 | Cam^{R}; pSC101 ori (ts) | Source of cam^{R}:sacB | 34 |
| | | cassette | |
| pREDTKI | Kan^{R}; pSC101 ori (ts); | Bifunctional λ Red | 32 |
| | araBAD promoter for | and I-SceI helper | |
| | λ Red genes; trc | plasmid | |
| | promoter for I-SceI | | |
| pKSI-I | Amp^{R}; pUC ori | pBluescript II KS(−) | 32 |
| | | backbone with I-SceI | |
| | | site-MCS-I-SceI site | |
| | | cassette | |
| pCM01 | Amp^{R}; pUC ori | pKSI-I + cam^{R}:sacB | This study |
| pCM02 | Amp^{R}; pUC ori | pKSI-1 + aceE | This study |
| pCM03 | Amp^{R}; pUC ori | pKSI-I + | This study |
| | | aceE::cam^{R}:sacB | |
| pCM04 | Amp^{R}; pUC ori | pKSI-I + aceE[H106M] | This study |
| pCM05 | Amp^{R}; pUC ori | pKSI-I + aceE[H106N] | This study |
| pCM06 | Amp^{R}; pUC ori | pKSI-I + aceE[H106V] | This study |
| pCM07 | Amp^{R}; pUC ori | pKSI-I + aceE[E401A] | This study |
| pCM08 | Amp^{R}; pUC ori | pKSI-I + aceE[E401D] | This study |
| pCM09 | Amp^{R}; pUC ori | pKSI-I + aceE[K403A] | This study |
| pCM10 | Amp^{R}; pUC ori | pKSI-I + aceE[K403N] | This study |
| pCM11 | Amp^{R}; pUC ori | pKSI-I + aceE[K403Q] | This study |
| pCM12 | Amp^{R}; pUC ori | pKSI-I + aceE[K410M] | This study |
| pCM13 | Amp^{R}; pUC ori | pKSI-I + aceE[K410N] | This study |
| pCM14 | Amp^{R}; pUC ori | pKSI-I + aceE[K410Q] | This study |
| pCM16 | Amp^{R}; pUC ori | pKSI-I + aceE[G395A] | This study |
| pCM17 | Amp^{R}; pUC ori | pKSI-I + aceE[H142V] | This study |
| pCM18 | Amp^{R}; pUC ori | pKSI-I + aceE[H640V] | This study |
| pCM19 | Amp^{R}; pUC ori | pKSI-I + aceE[Y177F] | This study |
| pCM21 | Amp^{R}; pUC ori | pKSI-I + | This study |
| | | aceE[H106M; E401A] | |

To construct pCM01, a cam-sacB cassette was amplified from pEL04 (34) and cloned into the MCS of pKSI-I (32). To construct pCM02, aceE with 500 bp of flanking DNA on both sides was cloned into the multiple cloning site (MCS) of pKSI-1. The cam-sacB cassette was amplified from pCM01 with I-SceI restriction sites incorporated into both primers, and cloned into pCM02, replacing bases 4-2,596 of the coding sequence of aceE, to generate pCM03. All plasmids harboring a single point-mutated aceE variant were generated from pCM02 using mutagenic primers that incorporated mutations into homologous regions used for DNA assembly. To create pCM21, which harbors two point mutations, pCM04 was used as a template to incorporate the mutation E401A. Codon usage frequency of E. coli was considered in the design of point mutations.

TABLE 3
Primers used in this study.

| Name | Description | Sequence 5′-3′ |
|---|---|---|
| MEP166 | IdhA F | TTAAGCATTCAATACGGGTATTGTG |
| MEP167 | IdhA R | GTCATTACTTACACATCCCGCCATC |
| MEP168 | aceE F | TGAGCGTTCTCTGCGTCGTCTGGA G |
| MEP169 | aceE R | ATCGCCAACAGAGACTTTGATCTC |
| MEP288 | poxB F | CCGGTTGTCGCTGCCTGC |
| MEP289 | poxB R | TTCAAACAGATAGTTATGCGCGGCC |
| MEP290 | ppsA F | CGCACAGAAGCGTAGAACGTTATG |
| MEP291 | ppsA R | CGTTTAGGTGAACGATCATGCGC |
| MEP398 | KSI-HA-ace-F | GACAAGGCTTCTATGGAAGCTGCAGGAATTCGATATCAAG |
| MEP399 | KSI-HA-ace-R | GAGTACGGCGTTTGATTCCGATCCACTAGTTCTAGAGCG |
| MEP400 | ace-HA-KSI-F | GCTCTAGAACTAGTGGATCGGAATCAAACGCCGTACTC |
| MEP401 | ace-HA-KSI-R | TTGATATCGAATTCCTGCAGCTTCCATAGAAGCCTTGTCG |
| MEP402 | cam-HA-KSI-F | CGCTCTAGAACTAGTGGATCTGTGACGGAAGATCACTTC |
| MEP403 | cam-HA-KSI-R | CTTGATATCGAATTCCTGCAGATCAAAGGGAAAACTGTCC |
| MEP404 | KSI-HA-cam-F | GACAGTTTTCCCTTTGATCTGCAGGAATTCGATATCAAG |
| MEP405 | KSI-HA-cam-R | GAAGTGATCTTCCGTCACAGATCCACTAGTTCTAGAGCG |
| MEP418 | ace-seq-1 | GAAATATCTGGAACACCGTGG |
| MEP419 | ace-seq-2 | CCAAAGGCAAAGCGACAG |
| MEP420 | ace-seq-3 | CTTACTATAAAGAAGACGAGAAAGGTC |
| MEP421 | aceE-HA-SCE-R | CACATAGGGATAACAGGGTAATCATGGGTTATTCCTTATC |
| MEP422 | aceE-HA-SCE-F | GGACAGTAGGGATAACAGGGTAATAAGTGGTTGCTGACGC |
| MEP423 | cam-HA-SCE-R | CCACTTATTACCCTGTTATCCCTACTGTCCATATGCACAG |
| MEP424 | cam-HA-SCE-F | CCCATGATTACCCTGTTATCCCTATGTGACGGAAGATCAC |
| MEP428 | aceE-H106M-F | AACTGGGCGGCATGATGGCGTCCTT |
| MEP429 | aceE-H106M-R | AAGGACGCCATCATGCCGCCCAGTT |
| MEP430 | aceE-H106V-F | AACTGGGCGGCGTGATGGCGTCCTT |
| MEP431 | aceE-H106V-R | AAGGACGCCATCACGCCGCCCAGTT |
| MEP432 | aceE-H106N-F | AACTGGGCGGCAACATGGCGTCCTT |
| MEP433 | aceE-H106N-R | AAGGACGCCATGTTGCCGCCCAGTT |
| MEP434 | aceE-E401A-F | GGCATGGGCGACGCGGCTGCAGGTAAAAACATCGCGCACC |
| MEP435 | aceE-E401A-R | GGTGCGCGATGTTTTTACCTGCAGCCGCGTCGCCCATGCC |
| MEP440 | aceE-E401D-F | GGCATGGGCGACGCGGCTGATGGTAAAAACATCGCGCACC |
| MEP441 | aceE-E401D-R | GGTGCGCGATGTTTTTACCATCAGCCGCGTCGCCCATGCC |
| MEP442 | aceE-K403A-F | GGCATGGGCGACGCGGCTGAAGGTGCAAACATCGCGCACC |
| MEP443 | aceE-K403A-R | GGTGCGCGATGTTTGCACCTTCAGCCGCGTCGCCCATGCC |
| MEP444 | aceE-K403N-F | GGCATGGGCGACGCGGCTGAAGGTAACAACATCGCGCACC |
| MEP445 | aceE-K403N-R | GGTGCGCGATGTTGTTACCTTCAGCCGCGTCGCCCATGCC |
| MEP446 | aceE-K403Q-F | GGCATGGGCGACGCGGCTGAAGGTCAGAACATCGCGCACC |
| MEP447 | aceE-K403Q-R | GGTGCGCGATGTTCTGACCTTCAGCCGCGTCGCCCATGCC |
| MEP448 | aceE-K410N-F | CATCGCGCACCAGGTTAACAAAATGAACATGGACG |
| MEP449 | aceE-K410N-R | CGTCCATGTTCATTTTGTTAACCTGGTGCGCGATG |
| MEP450 | aceE-K410Q-F | CATCGCGCACCAGGTTCAGAAAATGAACATGGACG |
| MEP451 | aceE-K410Q-R | CGTCCATGTTCATTTTCTGAACCTGGTGCGCGATG |
| MEP452 | aceE-K410M-F | CATCGCGCACCAGGTTATGAAAATGAACATGGACG |
| MEP453 | aceE-K410M-R | CGTCCATGTTCATTTTCATAACCTGGTGCGCGATG |
| MEP454 | aceE-G395A-F | GCTCATACCATTAAAGGTTACGCGATGGGCGACGCGGCTG |
| MEP455 | aceE-G395A-R | CAGCCGCGTCGCCCATCGCGTAACCTTTAATGGTATGAGC |
| MEP602 | aceE-H640V-F | AACGGCGAAGGTCTGCAGGTAGAAGATGGTCACAGCCAC |
| MEP603 | aceE-H640V-R | GTGGCTGTGACCATCTTCTACCTGCAGACCTTCGCCGTT |
| MEP604 | aceE-H142V-F | TACTTCCAGGGCGTAATCTCCCCG |
| MEP605 | aceE-H142V-R | CGGGGAGATTACGCCCTGGAAGTA |
| MEP655 | aceE-Y177F-F | GCAATGGCCTCTCTTCCTTCCCGCACCCGAAACTGATGC |
| MEP656 | aceE-Y177F-R | GCATCAGTTTCGGGTGCGGGAAGGAAGAGAGGCCATTGC |
| MEP852 | aceE-RT-F | GTATTGGCGATCTGTGCTGG |
| MEP853 | aceE-RT-R | CTGTGACCATCTTCGTGCTG |
| MEP856 | rpoD-RT-F | TGATGCTGGCTGAAAACACC |
| MEP857 | rpoD-RT-R | AGTTCAACGGTGCCCATTTC |

Media and Growth Conditions

During plasmid and strain construction, cultures were grown on LB while all aceE deletion mutants were grown in TYA medium. As needed, the following antibiotics were included in medium (final concentrations): ampicillin (100 μg/mL), kanamycin (40 μg/mL), and chloramphenicol (20 μg/mL). For counter-selection against sacB, the medium was supplemented with 250 g/L sucrose, and NaCl was excluded.

The defined basal medium to which carbon/energy sources were added contained (per L): 3.5 g NH₄Cl, 0.29 g KH₂PO₄, 0.50 K₂HPO₄.3H₂O, 2.0 g K₂SO₄, 0.45 g MgSO₄.7H₂O, 0.25 mg ZnSO₄.7H₂O, 0.125 mg CuCl₂.2H₂O, 1.25 mg MnSO₄—H₂O, 0.875 mg CoCl₂.6H₂O, 0.6 mg H₃BO₃, 0.25 mg Na₂MoO₄.2H₂O, 5.5 mg FeSO₄.7H₂O, 20 mg Na₂EDTA.2H₂O, 20 mg citric acid, 20 mg thiamine HCl, and 20.9 g 3-[N-morpholino]propanesulfonic acid (100 mM). Thiamine was filtered sterilized, while other medium components were autoclaved in compatible mixtures, combined and then adjusted to a pH of 7.1 with 20% (w/v) NaOH.

Shake Flask Experiments

A single colony from an LB plate was used to inoculate 3 mL TYA. After 6-10 h of growth, this culture was used to inoculate 3 mL of basal medium with 5 g/L D-(+)-glucose to an initial optical density at 600 nm (OD) of 0.5. After 12-15 h of growth, this culture was used to inoculate three 250 mL baffled shake flasks containing 50 mL of basal medium with 5 g/L glucose to an OD of 0.2. All cultures were grown aerobically at 37° C. on a rotary shaker at 225 rpm. Flasks were sampled 6-8 times for measurement of growth rate and extracellular metabolite concentrations.

Batch and Repeated Batch Processes

A single colony from an LB plate was used to inoculate 3 mL TYA. After 6-10 h of growth, this culture was used to inoculate a 250 mL shake flask containing 50 mL of basal medium with 5 g/L glucose or 5 g/L glucose plus 2.34 g/L Na(CH₃COO).3H₂O (1 g/L acetate) to an OD of 0.2. When the shake flask culture reached an OD of 2, the entire 50 mL contents were used to inoculate a 2.5 L bioreactor (Bioflo 2000, New Brunswick Scientific Co., New Brunswick, N.J., USA) containing 950 mL basal medium with 15 g/L glucose. Duplicate batch processes were performed.

A repeated batch process started as a 1.0 L batch process described above containing 15 g/L glucose and 2 g/L acetate. When glucose was just depleted, a 17 mL solution containing glucose was added to increase its concentration nominally to 15 g/L. Batch and repeated batch studies were conducted with a constant agitation of 400 rpm and at 37° C. Air and/or oxygen-supplemented air was sparged at 1.0 L/min to maintain a dissolved oxygen concentration above 40% of saturation. The pH was controlled at 7.0 using 25% (w/v) KOH/5% NH₄OH and 20% (w/v) H₂SO₄. Antifoam C (Sigma) was used as necessary to control foaming.

Continuous Process

Nitrogen-limited steady-state processes were conducted as chemostats and started as a 0.5 L batch process containing basal medium with 15 g/L glucose and the following modifications: NH₄Cl concentration was reduced from 3.5 g/L to 1 g/L and 3-[N-morpholino]propanesulfonic acid concentration was reduced from 20.9 g/L to 10.5 g/L (50 mM). Each strain was initially cultured in TYA and then inoculated into a 150 mL baffled shake flask containing 25 mL of modified basal medium with 15 g/L glucose. When the shake flask culture reached an OD of 2, the entire 25 mL contents were used to inoculate a 1.0 L bioreactor (Bioflo 310, New Brunswick Scientific Co., New Brunswick, N.J., USA) containing 475 mL modified basal medium with 15 g/L glucose. After growth to an OD of 4, the chemostat was initiated at a nominal dilution rate of 0.15 h⁻¹, 0.20 h⁻¹, or 0.28 h⁻¹. The influent medium contained modified basal medium with 15 g/L glucose, and pH was adjusted to 8 to facilitate the control of pH within the bioreactor (at 7.0). The process operated at 37° C. using 400 rpm agitation. Air and/or oxygen-supplemented air was sparged at 0.5 L/min to maintain a dissolved oxygen concentration above 40% of saturation. The pH was controlled at 7.0 with 30% w/v KOH, and antifoam C (Sigma) was used as necessary to control foaming.

Analytical Methods

The optical density at 600 nm (OD) (UV-650 spectrophotometer, Beckman Instruments, San Jose, Calif., USA) was used to monitor cell growth. For dry cell weight measurement, three 20.0 mL samples were centrifuged (3300×g, 10 min), the pellets washed by vortex mixing with 20 mL DI water and then centrifuged again. After washing three times, the cell pellets were dried at 60° C. for 24 h and weighed. Samples were routinely frozen at −20° C. for further analysis, and thawed samples were centrifuged (4° C., 10000×g for 10 min), and filtered (0.45 μm nylon, Acrodisc, Pall Corporation, Port Washington, N.Y.). Liquid chromatography was used to quantify pyruvate, glucose and organic products using RI detection (36). Ammonium was quantified by the Laboratory for Environmental Analysis (University of Georgia, Athens, Ga., USA) using the phenate method (37).

RT-qPCR

Total RNA was prepared from chemostat samples (10⁹ cells) at each dilution rate using Monarch Total RNA Miniprep Kit (New England Biolabs, Ipswich, Mass., USA). RNA was used as a template for PCR to confirm that no genomic DNA was remaining. One-Step quantitative reverse transcriptase PCR (RT-qPCR) was performed using Luna Universal One-Step RT-qPCR Kit (New England Biolabs, Ipswich, Mass., USA) on a StepOne Plus instrument (Applied Biosystems, Foster City, Calif.). Primer pairs for aceE and the housekeeping gene rpoD were confirmed to have similar efficiencies. Triplicate 20 μL reactions containing 4 ng total RNA were analyzed. No-template and no-RT controls were included. The 2^{-ΔΔCT} method was used to calculate fold change in expression from C_{T} values generated by the StepOne Plus software (38).

### Results

Variant Strain Screening

The principal goal of this proof-of-concept study was to generate point mutations in the E1 component of PDHc coded by aceE to decrease the activity of this enzyme and thereby reduce the flux from pyruvate to acetyl-CoA. Certain mutations, such as those critical in mediating the reaction or those integral to structure, would likely inactivate PDHc, eliminate the flux, and prevent growth of the strain on glucose as the sole carbon source. Our hypothesis was that other less severe mutations would permit growth but accumulate pyruvate, the substrate for that enzyme. To prevent the conversion of pyruvate to the typical by-products lactate and acetate, the IdhA and poxB genes were deleted in all strains (22, 23).

The E1 subunit of the PDHc in E. coli consists of an AceE homodimer containing two symmetrical active site clefts that are formed at the interface of the two monomers. These active site domains catalyze the ThDP-dependent decarboxylation of pyruvate to an intermediate, 2α-hydroxyethylidene-ThDP, and subsequently acetylates lipoate moieties of the E2 subunit (26, 39, 40). Previous studies and protein structure were used to propose target residues in AceE involved in active site formation and pyruvate binding that might reduce activity without eliminating it (41-43). In particular, we targeted 1) the residues lining the active site cleft, and 2) the inner active site loop, one of two dynamic loops that gate the active site and interact with the E2 subunit (41). In the active site cleft lining, three histidine residues and one tyrosine residue were targeted for mutagenesis: H106, H142, and H640 are proximal to the reactive C2 atom of the thiazolium ring of ThDP where they are predicted to orient pyruvate in the active site, while Y177 is predicted to interact with ThDP intermediates (42). The inner mobile loop is comprised of residues 401-413 flanked on the N-terminal side by three glycine residues (41). In this region, G395, E401, K403, and K410 were targeted for mutagenesis. E401 and K403 stabilize the loop by forming hydrogen bonds with G395 and other adjacent residues, while K410 assists in ThDP entry into the active site cleft. These 8 residues were mutated to generate 16 AceE variants (15 single-point mutations, 1 double-point mutation) incorporated into the E. coli chromosome.

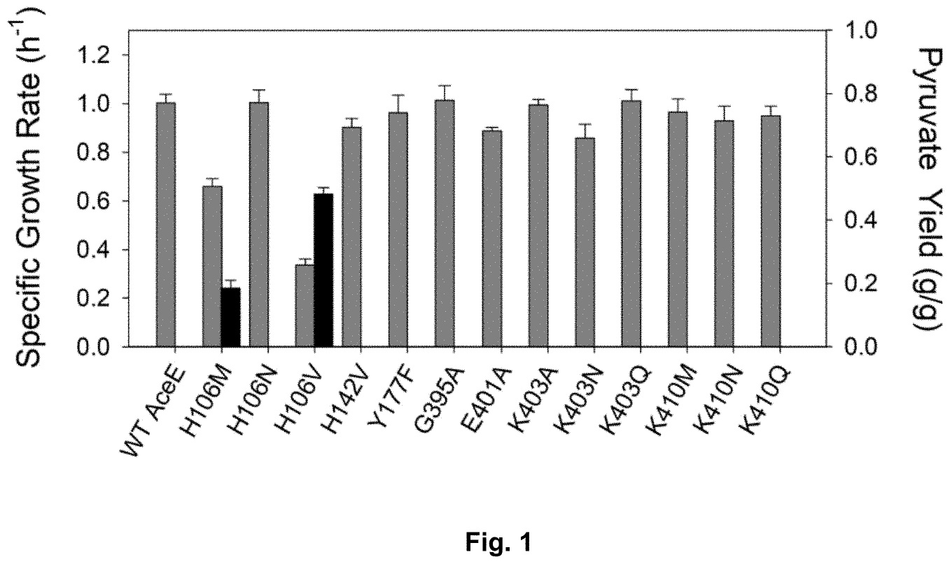

In order to determine whether each selected AceE mutation altered central metabolism, the single-point mutation variants were examined in triplicate in shake flasks for specific growth rate and pyruvate yield (FIG. 1). Of the 15 strains with single-point mutations, 13 showed growth on glucose as the sole carbon source. MEC861 (AceE[E401 D]) and MEC919 (AceE[H640V]) did not grow after 24 h. MEC860 (AceE[H106M]) and MEC826 (AceE[H106V]) were the only variants that accumulated pyruvate at yields of 0.19±0.3 g/g (MEC860) and 0.48±0.2 g/g (MEC826). The accumulation of pyruvate correlated with a decrease in growth rate. Compared to MEC825 expressing the wild-type AceE (1.00±0.4 h⁻¹), the H106M variant attained a growth rate of 0.66±0.3 h⁻¹, while the H106V variant attained a growth rate of 0.34±0.3 h⁻¹. All other variants attained growth rates above 0.86 h⁻¹. We also constructed strain MEC956 containing two mutations (AceE[H106M;E401A]). MEC956 attained a growth rate of 0.4 h⁻¹, 96% slower than MEC825, and a 0.86 g/g pyruvate yield in shake flasks. MEC826 (AceE[H106V]) attained the highest pyruvate yield at a reasonable growth rate and was chosen for further studies.

Effect of ppsA Inactivation in Pyruvate-Accumulating Strains

PEP synthase encoded by ppsA converts pyruvate into PEP and is essential when pyruvate is used as the sole carbon source (44, 45). When MEC826 was grown in shake flask, pyruvate was slowly consumed after glucose was depleted (data not shown). This result suggested that these variants have a route to assimilate pyruvate, and we suspected PEP synthase was the cause. To minimize pyruvate utilization, and potentially increase its yield, ppsA was inactivated in MEC826 to yield MEC905 (C aceE::aceE^{[H106V]} IdhA poxB ppsA). We also constructed MEC961 (C aceE::aceE IdhA poxB ppsA) expressing the wild-type AceE. These two strains were grown in triplicate in shake flasks as before to determine specific growth rate and pyruvate yield. MEC961 grew at a specific growth rate of 1.4±0.00 h⁻¹ and did not accumulate pyruvate, identical to results observed with MEC825. In contrast, MEC905 attained a specific growth rate of 0.32±0.01 h⁻¹ and accumulated pyruvate at a yield of 0.52±0.2 g/g. Compared to the strain with ppsA (MEC826), MEC905 exhibited a small decrease in growth rate and increase in pyruvate yield.

Controlled Batch Processes

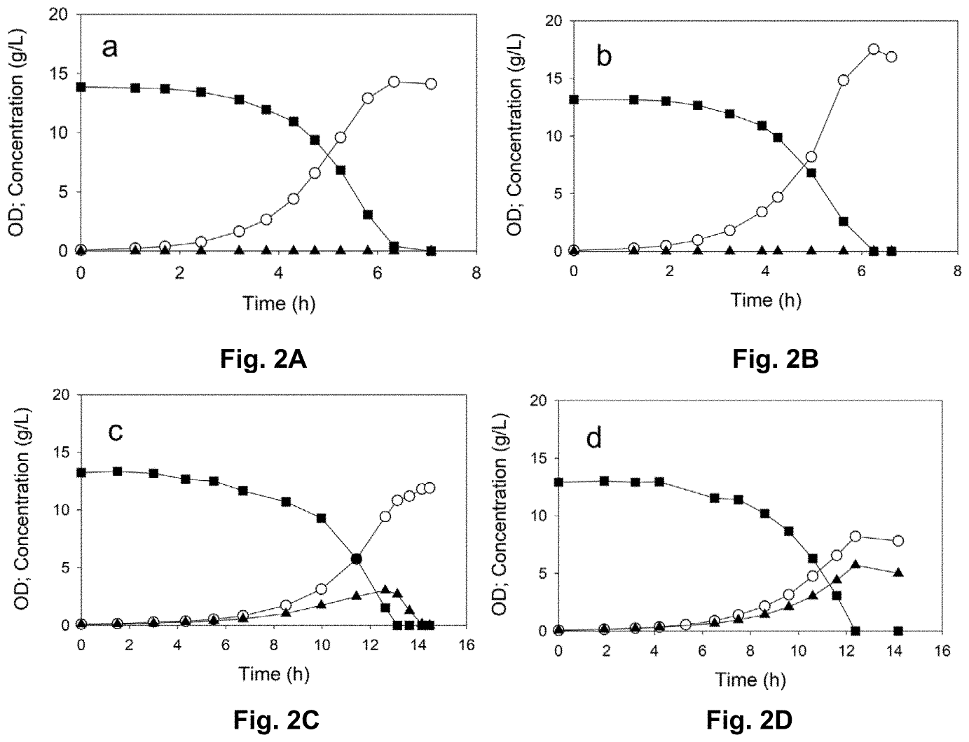

Our initial comparison of AceE variants was conducted in shake flasks in which oxygenation and pH were not well-controlled. We therefore compared growth and pyruvate formation using MEC825, MEC826, MEC905, or MEC961 in controlled 1.0 L bioreactors using 15 g/L glucose as the sole carbon source (FIG. 2). All four strains contain knockouts in IdhA and poxB. MEC825 and MEC961 express the wild-type AceE, while MEC826 and MEC905 express the variant AceE[H106V]. The strains differ in having an intact ppsA gene (MEC825 and MEC826) or a ppsA deletion (MEC905 and MEC961). Both MEC825 (FIG. 2a) and MEC961 (FIG. 2b) depleted the glucose in less than 7 h and attained a growth rates of 0.93-0.99 h⁻¹. Neither strain accumulated pyruvate. MEC826 achieved a pyruvate yield of 0.29±0.5 g/g (FIG. 2c), while MEC905 achieved a yield of 0.43±0.01 g/g (FIG. 2d). These two strains expressing the AceE[H106V] variant showed no significant difference in growth rate (0.39±0.01 h⁻¹). MEC826 metabolized pyruvate within two hours after glucose was depleted, whereas MEC905 metabolized pyruvate slowly after glucose depletion, demonstrating that pyruvate assimilation due to the activity of PEP synthase is significant.

Steady-State Process

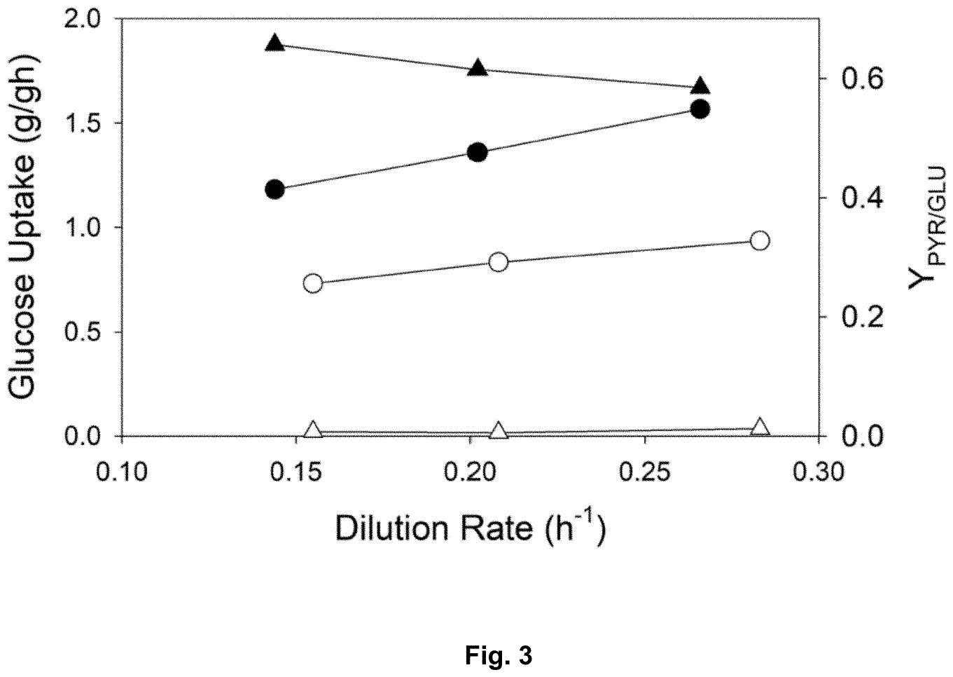

During batch growth all nutrients are in excess for essentially the entire process. In contrast, a steady-state process conducted as a chemostat permits the selection of one growth-limiting nutrient. Because the accumulation of pyruvate and other carbon products is often enhanced by carbon-excess conditions (46), we examined pyruvate accumulation using MEC905 and MEC961 at nominal growth rates of 0.15, 0.20, and 0.28 h⁻¹ under nitrogen limitation (FIG. 3, Table 4). Nitrogen limitation was confirmed (as opposed to oxygen limitation) by 1) maintaining the DO above 30% saturation, 2) the absence of anaerobic products in the effluent such as formate, 3) the presence of glucose in the effluent, and 4) the absence of nitrogen in the effluent. Additionally, we examined aceE expression levels in each strain at the target dilution rates. These strains both contain IdhA, poxB, and ppsA knockouts, but MEC905 expresses the AceE[H106V] variant and MEC961 expresses wild-type AceE. After five residence times to achieve steady-state at each of the three dilution rates, MEC905 accumulated pyruvate at an average yield of 0.62±0.4 g/g with an average specific productivity of 0.84±0.7 g/gh. At each dilution rate MEC961 accumulated insignificant pyruvate (average yield of only 0.01±0.00 g/g). In contrast, MEC905 did not accumulate acetate, while MEC961 accumulated acetate at an average yield of 0.11±0.01 g/g and specific productivity of 0.9±0.2 g/gh. At each dilution rate the specific glucose consumption rate of MEC905 (average 1.37±0.19 g/gh) was much greater than that of MEC961 (0.83±0.10 g/gh). Between the two strains at three dilution rates, aceE was upregulated 2.61±0.54 fold in MEC905 compared to MEC961 (Table 5).

TABLE 4
Chemostat results. Cultures were grown in 0.5 liters of a nitrogen-limited
defined medium with nominally 15 g/L glucose as the sole carbon source. After
3-4 residence times to achieve a steady-state, the feed and effluent were
measured for the concentrations of organic compounds and the ammonium ion.

| | | Feed | | Effluent | | | | |
|---|---|---|---|---|---|---|---|---|
| | D | Glucose | N | Glucose | N | Biomass | Acetate | Pyruvate |
| Strain | (h⁻¹) | (g/L) | (mg/L) | (g/L) | (mg/L) | (dry g/L) | (g/L) | (g/L) |
| MEC905 | 0.144 | 14.9 | 213 | 3.9 | 0.0 | 1.43 | 0.00 | 7.73 |
| MEC905 | 0.202 | 15.0 | 187 | 4.79 | 0.1 | 1.52 | 0.00 | 6.29 |
| MEC905 | 0.266 | 14.7 | 247 | 5.84 | 0.0 | 1.50 | 0.00 | 5.18 |
| MEC961 | 0.155 | 14.2 | 236 | 4.84 | 0.0 | 1.97 | 0.91 | 0.7 |
| MEC961 | 0.208 | 14.2 | 236 | 6.39 | 0.1 | 1.94 | 0.82 | 0.5 |
| MEC961 | 0.283 | 14.2 | 236 | 7.70 | 0.3 | 1.95 | 0.72 | 0.8 |

TABLE 5
RT-qPCR results from chemostat experiments. One-step quantitative reverse transcriptase
PCR was performed on samples taken for each dilution rate at steady-state.

| | D | Average C_{T} | | ΔC_{T} | | aceE |
|---|---|---|---|---|---|---|
| Strain | (h⁻¹) | aceE | rpoD | aceE − rpoD | ΔΔC_{T} | Fold Change |
| MEC905 | 0.144 | 26.95 ± 0.9 | 25.21 ± 0.6 | 1.74 ± 0.11 | −1.7 ± 0.11 | 2.10 |
| | | | | | | (1.94-2.27) |
| MEC905 | 0.202 | 26.15 ± 0.3 | 24.70 ± 0.5 | 1.45 ± 0.6 | −1.36 ± 0.6 | 2.56 |
| | | | | | | (2.45-2.67) |
| MEC905 | 0.266 | 26.46 ± 0.11 | 25.10 ± 0.6 | 1.37 ± 0.13 | −1.66 ± 0.13 | 3.17 |
| | | | | | | (2.90-3.45) |
| MEC961 | 0.155 | 28.66 ± 0.14 | 25.85 ± 0.7 | 2.81 ± 0.15 | 0.00 ± 0.15 | 1.00 |
| | | | | | | (0.85-1.15) |
| MEC961 | 0.208 | 27.75 ± 0.10 | 24.70 ± 0.5 | 3.6 ± 0.12 | 0.00 ± 0.12 | 1.00 |
| | | | | | | (0.88-1.12) |
| MEC961 | 0.283 | 27.23 ± 0.3 | 24.20 ± 0.8 | 3.3 ± 0.8 | 0.00 ± 0.8 | 1.00 |
| | | | | | | (0.92-1.8) |

Pyruvate Production by H106M/E401A Variant Strains

Of the AceE variants examined, MEC956 was the only strain that contained two point mutations (AceE[H106M;E401A]). This strain grew very poorly using glucose as the sole carbon source (0.4 h⁻¹), but it attained a pyruvate yield exceeding 0.80 g/g in shake flasks, higher than previously reported (47). This result suggests a two-phase process in which the strain is grown initially on complex carbon sources or on a carbon source biochemically downstream of PDHc to supply acetyl CoA (22, 23). To prevent pyruvate assimilation by MEC956, ppsA was inactivated to generate MEC994 (C aceE::aceE^{[H106M;E401A]} IdhA poxB ppsA). The performance of MEC994 was compared to MEC992 (C aceE IdhA poxB ppsA) which contains an aceE knockout.

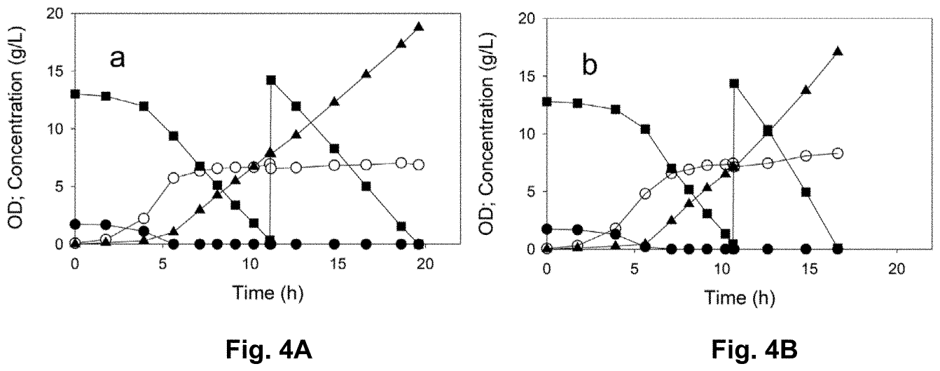

MEC992 and MEC994 were each grown under controlled batch conditions at 1.0 L scale initially containing 15 g/L glucose and 2 g/L acetate, with 15 g glucose added once after the glucose was depleted. As expected, growth of MEC992 ceased after acetate depletion, while MEC994 sustained a slow growth rate (FIG. 4). After acetate depletion, MEC992 accumulated pyruvate at a yield of 0.77 g/g and volumetric productivity of 1.25 g/Lh, and converted the added glucose to pyruvate at a yield of 0.78 g/g and volumetric productivity of 1.28 g/Lh (FIG. 4a). By the end of the process, MEC992 generated 18.8 g/L in 19.5 h for an overall yield of 0.73 g/g and productivity of 0.96 g/Lh (includes growth). In comparison, MEC994 converted the initial glucose to pyruvate at 0.73 g/g yield with a volumetric productivity of 1.32 g/Lh, and converted the added glucose at 0.74 g/g yield with a volumetric productivity of 1.70 g/Lh (FIG. 4b). At the end of the process, MEC994 accumulated 17.1 g/L pyruvate in 16.6 h for an overall yield of 0.68 g/g and productivity of 1.3 g/Lh. Thus, under twice-repeated batch conditions the aceE deletion strain MEC992 provided a slightly greater yield but a lower productivity. Importantly, pyruvate productivity for MEC994 increased by 29% between the first and second period of glucose consumption, while the pyruvate productivity was essentially unchanged for MEC992. This result suggests that slow growth during pyruvate formation could better maintain pyruvate production during a prolonged process with glucose as the sole carbon source.

During all batch and continuous processes, no significant unknown peaks were observed and additional by-products (e.g., succinate, ethanol, citrate, glyoxylate, formate, etc.) were less than 0.1 g/L.

Discussion

In this proof-of-concept study, point mutations in AceE were engineered into the chromosome of E. coli, and the strains were screened for their ability to accumulate pyruvate. The goal was to use a structure-based approach to engineer AceE variants having reduced activity, such that pyruvate conversion to acetyl CoA by PDHc was greatly diminished but not eliminated. By considering the pyruvate binding domain of AceE and previous structural studies, we mutated residues involved in active site formation and substrate binding (41-43).

The H106 mutations were the only mutation site which allowed pyruvate accumulation (H106M and H106V), and resulted in a >15% decrease in growth rates. These mutations are most likely inhibiting pyruvate reacting with the thiazolium ring of ThDP and/or decreasing the stability of reaction intermediates. Although the most likely explanation for pyruvate accumulation and reduced growth rate is that these mutations affect the kinetic properties of the enzyme, we cannot rule out reduced protein stability or altered regulation. Eight of the nine variants with substitutions in mobile loop residues (G395, E401, K403, and K410) had a comparatively minor impact on growth rate and did not accumulate pyruvate in shake flask culture. These results suggest that the mobile loop in these variants was marginally impaired and did not impact PDHc activity sufficiently to accumulate pyruvate. Interestingly, a few variants that were previously reported to have very low in vitro activity did not lead to pyruvate accumulation. For example, Y177F, E401A, and K403A have been previously shown to reduce PDHc activities by 93%, 90%, and 88%, respectively (41, 43), yet strains chromosomally expressing these mutations displayed only a 5-15% decrease in growth rate on glucose with no pyruvate accumulation (FIG. 1). Thus, even a considerable decrease in enzyme activity does not predict pyruvate accumulation. These results can be explained by the differences between in vitro and in vivo environments and the fact that in vitro assays do not replicate the intracellular environment (48, 49). Additionally, a relationship was observed between a strain's specific growth rate and pyruvate yield: the lower the growth rate, the greater the pyruvate yield. A threshold growth rate appears to exist for pyruvate accumulation: E401A and K403N (growth rate of 0.86-0.89 h⁻¹, about 15% lower than MEC825) did not accumulate pyruvate while H106M (0.66 h⁻¹) did.

Two variants examined (E401 D, H640V) were not able to grow on glucose as a sole carbon source, indicating these substitutions were too deleterious to allow sufficient PDHc activity required for growth. E401 likely contributes to loop stability by hydrogen bonding with proximal glycine residues, and a charge reversal substitution (E401K) has been shown to impair mobile loop function (41). The E401D substitution likely disrupts stabilizing hydrogen bonds similar to the E401K substitution, as the side chain carboxyl of E401 D is oriented away from the original position where undesirable hydrogen bonds can decrease loop stability. In comparison, the fact that E401A only reduced growth rate slightly (FIG. 1) demonstrates that the specific substitution is critical to the enzyme. H640 is located in the active site cleft and likely is involved in the reaction between pyruvate and ThDP to stabilize the reaction intermediates (42). Since the H640V variant could not utilize glucose as a carbon source, this mutation may inhibit the formation and stability of intermediates.

MEC956 contained one mutation in the active site (H106M) and one mutation in the mobile loop (E401A). This strain attained the highest pyruvate yield in shake flask culture but grew very slowly, much slower than MEC860 (H106M alone), despite the observation that MEC827 (E401A alone) resulted in only a modest decline in growth rate with no pyruvate generation. Clearly, the combination of mutations causes a synergistic effect on enzyme activity.

Since growing MEC956 on glucose as the sole carbon source would be impractical due to its slow growth rate, a two-phase process was used where acetate was supplied in the initial phase to support biomass formation. Using this process, MEC994 (C aceE::aceE^{[H106M;E401A]} poxB IdhA ppsA) was effective in generating pyruvate after acetate was consumed. When compared to MEC992 (C aceE poxB IdhA ppsA), the diminished activity of AceE[H106M;E401A] allowed MEC994 to grow on glucose, leading to a slight decrease in pyruvate yield but an increase in volumetric productivity. The AceE[H106M;E401A] variant is a similar alternative to inactivating aceE to increase and prolong the productivity of pyruvate-producing strains.

The observed inverse correlation between growth rate and pyruvate accumulation emphasizes the competition between cell growth and product formation. In pyruvate-accumulating strains, the decreased activity of PDHc reduced the rate of acetyl CoA formation, and carbon flux was diverted from central metabolism to pyruvate accumulation. This balance is further evident when comparing MEC905 (AceE[H106V]) and MEC961 (wild-type AceE) under nitrogen-limited steady-state conditions. On a per cell basis and considering the three different dilution rates, MEC905 consumed glucose 65% faster and sustained 2.6-fold greater aceE expression levels than MEC961 in order to maintain the target growth rates and presumably equivalent acetyl CoA formation rates needed for biomass formation.

By-product pathways to lactate and acetate were effectively blocked by the deletion of the IdhA and poxB genes (22, 23), and neither lactate nor acetate (nor other typical by-products) was observed in cultures during growth of any variant in shake flasks or batch processes. Pyruvate consumption was observed when glucose was depleted, an effect that was largely eliminated by the ppsA deletion. The ppsA deletion therefore resulted in much greater pyruvate yield with limited effect on growth rate.

Of the 12 precursors generated in central metabolism during aerobic growth of wild-type E. coli on glucose, pyruvate ranks second on a molar basis in the quantity withdrawn for biomass, with over 86% of the carbon entering pyruvate from PEP exiting through PDHc (24). The kinetic parameters of E. coli AceE, the rate-limiting component of the complex, for pyruvate are k_{cat} of 38 s⁻¹ and Kₘ of 260 mM (41). Any other enzyme overexpressed in an attempt to generate a product derived from pyruvate must directly compete with AceE. For example, acetolactate synthase (ALS) leading to the formation of isobutanol and valine has kinetic parameters of k_{cat} of 121 s⁻¹ and Kₘ of 13,600 mM, although the Q487S point mutation on ALS alters those parameters to k_{cat} of 11 s⁻¹ and Kₘ of 1,100 mM (50). The intracellular pyruvate concentration under aerobic conditions when E. coli grows maximally is about 5,000 mM (51, 52). Assuming this pyruvate concentration, AceE would operate near capacity (i.e., considering Michaelis-Menten kinetics—38×5000/(260+5000)=36 s⁻¹), while the seemingly faster wild-type ALS would be at 25% capacity (30 s⁻¹). Considering the case where the same quantity of these two enzymes are present without additional affectors, ALS flux is predicted to be 20% lower than the flux through PDHc despite having a k_{ca}t which is 3.2-fold greater. These simplified calculations serve to emphasize that enzyme activity is based both on the enzyme turnover (k_{cat}) and substrate binding (Kₘn), and that a high Kₘ limits the effectiveness of the enzyme, which itself depends on growth conditions. Unsurprisingly, enzymes like AceE found in central metabolism tend to have low values for Kₘ, which stands as a hurdle for a pathway toward any biochemical which must compete with these enzymes.

Continuing this example comparing AceE and ALS, merely expressing more ALS unfortunately becomes self-limiting since the presence of more ALS would tend to reduce pyruvate levels as has been observed with other enzymes (53), allowing AceE with its much lower Kₘ to become even more competitive. As an illustration, when wild-type E. coli is growing at 0.1 h⁻¹ the intracellular pyruvate has diminished to 1,500 mM (52), a level at which AceE is operating at 32 s⁻¹ (85% capacity) while at this concentration wild-type ALS would be operating only at 12 s⁻¹ (10%), necessitating 2.7-fold expression more than PDHc to allow pyruvate to partition equally between the two enzymes. Decreased expression of PDHc, for example, by using weak promoters to drive expression of aceE (28), can effectively decrease Vₘₐₓ, but does not affect the intrinsic kinetic properties of AceE (k_{cat} and Kₘ). Modification of native enzymes at key junctures in metabolism offers an additional degree of flexibility in pathway engineering.

In summary, reducing the activity of PDHc through point mutations is a means to accumulate pyruvate during E. coli growth on glucose as the sole carbon source. Reduced PDHc activity simultaneously leads to a reduction in growth rate. This metabolic engineering strategy offers an additional approach in the toolbox to redirect carbon toward biochemical products.

### EXAMPLE 1 REFERENCES

1. Koffas M A, Jung G Y, Stephanopoulos G. 2003. Engineering metabolism and product formation in Corynebacterium glutamicum by coordinated gene overexpression. Metab Eng 5:32-41.
2. Aristidou A A, San K Y, Bennett G N. 1994. Modification of central metabolic pathway in Escherichia coli to reduce acetate accumulation by heterologous expression of the Bacillus subtilis acetolactate synthase gene. Biotechnol Bioeng 44:944-951.
3. Alper H, Fischer C, Nevoigt E, Stephanopoulos G. 2005. Tuning genetic control through promoter engineering. Proc Natl Acad Sci USA 102:12678-12683.
4. Causey T, Zhou S, Shanmugam K, Ingram L. 2003. Engineering the metabolism of Escherichia coli W3110 for the conversion of sugar to redox-neutral and oxidized products: homoacetate production. Proc Natl Acad Sci USA 100:825-832.
5. Martinez J A, Bolivar F, Escalante A. 2015. Shikimic acid production in Escherichia coli: from classical metabolic engineering strategies to omics applied to improve its production. Front Bioeng Biotechnol 3:145.
6. Floras N, Xiao J, Berry A, Bolivar F, Valle F. 1996. Pathway engineering for the production of aromatic compounds in Escherichia coli. Nat Biotechnol 14:620.
7. Wu X, Eiteman M A. 2016. Production of citramalate by metabolically engineered Escherichia coli. Biotechnol Bioeng 113:2670-2675.
8. Knop D R, Draths K, Chandran S S, Barker J L, von Daeniken R, Weber W, Frost J. 2001. Hydroaromatic equilibration during biosynthesis of shikimic acid. J Am Chem Soc 123:10173-10182.
9. Yokota A, Takao S. 1989. Pyruvic acid production by lipoic acid auxotrophs of Enterobacter aerogenes. Agric Biol Chem 53:705-711.
10. Yokota A, Shimizu H, Terasawa Y, Takaoka N, Tomita F. 1994. Pyruvic acid production by a lipoic acid auxotroph of Escherichia coli W1485. Appl Microbiol Biotechnol 41:638-646.
11. Yonehara T, Miyata R. 1994. Fermentative production of pyruvate from glucose by Torulopsis glabrata. J Ferment Bioeng 78:155-159.
12. Kubicek C, Röhr M. 1977. Influence of manganese on enzyme synthesis and citric acid accumulation in Aspergillus niger. European J Appl Microbiol 4:167-175.
13. Papagianni M. 2007. Advances in citric acid fermentation by Aspergillus niger: biochemical aspects, membrane transport and modeling. Biotechnol Adv 25:244-263.
14. Ikeda M, Katsumata R. 1992. Metabolic engineering to produce tyrosine or phenylalanine in a tryptophan-producing Corynebacterium glutamicum strain. Appl Environ Microbiol 58:781-785.
15. Colon G, Nguyen T, Jetten M, Sinskey A, Stephanopoulos G. 1995. Production of isoleucine by overexpression of ilvA in a Corynebacterium lactofermentum threonine producer. Appl Microbiol Biotechnol 43:482-488.
16. Holetko J, Elišáková V, Prouza M, Sobotka M, Nešvera J, Pátek M. 2009. Metabolic engineering of the L-valine biosynthesis pathway in Corynebacterium glutamicum using promoter activity modulation. J Biotechnol 139:203-210.
17. Kim J, Copley S D. 2012. Inhibitory cross-talk upon introduction of a new metabolic pathway into an existing metabolic network. Proc Natl Acad Sci USA 109:E2856-E2864.
18. Bhattacharya S K, Dubey A K. 1995. Metabolic burden as reflected by maintenance coefficient of recombinant Escherichia coli overexpressing target gene. Biotechnol Lett 17:1155-1160.
19. Dong H, Nilsson L, Kurland C G. 1995. Gratuitous overexpression of genes in Escherichia coli leads to growth inhibition and ribosome destruction. J Bacteriol 177:1497-1504.
20. Maleki N, Eiteman M A. 2017. Recent progress in the microbial production of pyruvic acid. Fermentation 3:8.
21. Atsumi S, Hanai T, Liao J C. 2008. Non-fermentative pathways for synthesis of branched-chain higher alcohols as biofuels. Nature 451:86.
22. Zelić B, Gerharz T, Bott M, Vasić-Rački [symbol] , Wandrey C, Takors R. 2003. Fed-batch process for pyruvate production by recombinant Escherichia coli YYC202 strain. Eng Life Sci 3:299-305.
23. Tomar A, Eiteman M, Altman E. 2003. The effect of acetate pathway mutations on the production of pyruvate in Escherichia coli. Appl Microbiol Biotechnol 62:76-82.
24. Zhao J, Baba T, Mori H, Shimizu K. 2004. Effect of zwf gene knockout on the metabolism of Escherichia coli grown on glucose or acetate. Metab Eng 6:164-174.
25. Langley D, Guest J. 1978. Biochemical Genetics of the α-Keto Acid Dehydrogenase Complexes of Escherichia coli K 12: Genetic Characterization and Regulatory Properties of Deletion Mutants. J Gen Microbiol 106:103-117.
26. Bisswanger H. 1974. Regulatory Properties of the Pyruvate-Dehydrogenase Complex from Escherichia coli: Thiamine Pyrophosphate as an Effector. Eur J Biochem 48:377-387.
27. Schmincke-Ott E, Bisswanger H. 1981. Dihydrolipoamide Dehydrogenase Component of the Pyruvate Dehydrogenase Complex from Escherichia coli K12: Comparative Characterization of the Free and the Complex-Bound Component. Eur J Biochem 114:413-420.
28. Buchholz J, Schwentner A, Brunnenkan B, Gabris C, Grimm S, Gerstmeir R, Takors R, Eikmanns B J, Blombach B. 2013. Platform engineering of Corynebacterium glutamicum with reduced pyruvate dehydrogenase complex activity for improved production of L-lysine, L-valine, and 2-ketoisovalerate. Appl Environ Microbiol 79:5566-5575.
29. Datsenko K A, Wanner B L. 2000. One-step inactivation of chromosomal genes in Escherichia coli K-12 using PCR products. Proc Natl Acad Sci USA 97:6640-6645.
30. Baba T, Ara T, Hasegawa M, Takai Y, Okumura Y, Baba M, Datsenko K A, Tomita M, Wanner B L, Mori H. 2006. Construction of Escherichia coli K-12 in-frame, single-gene knockout mutants: the Keio collection. Mol Syst Biol 2:2006.0008
31. Chang Y-Y, Cronan J E. 1982. Mapping nonselectable genes of Escherichia coli by using transposon Tn10: location of a gene affecting pyruvate oxidase. J Bacteriol 151:1279-1289.
32. Yang J, Sun B, Huang H, Jiang Y, Diao L, Chen B, Xu C, Wang X, Liu J, Jiang W. 2014. High-efficiency scarless genetic modification in Escherichia coli by using lambda red recombination and I-SceI cleavage. Appl Environ Microbiol 80:3826-3834.
33. Thomason L C, Sawitzke J A, Li X, Costantino N, Court D L. 2014. Recombineering: genetic engineering in bacteria using homologous recombination. Curr Protoc Mol Biol 106:1.16.1-1.16. 39.
34. Lee E-C, Yu D, De Velasco J M, Tessarollo L, Swing D A, Court D L, Jenkins N A, Copeland N G. 2001. A highly efficient Escherichia coli-based chromosome engineering system adapted for recombinogenic targeting and subcloning of BAC DNA. Genomics 73:56-65.
35. Kostylev M, Otwell A E, Richardson R E, Suzuki Y. 2015. Cloning should be simple: Escherichia coli DH5α-mediated assembly of multiple DNA fragments with short end homologies. PLoS One 10:e0137466.
36. Eiteman M, Chastain M. 1997. Optimization of the ion-exchange analysis of organic acids from fermentation. Anal Chim Acta 338:69-75.
37. Solórzano L. 1969. Determination of ammonia in natural waters by the phenolhypochlorite method. Limnol Oceanogr 14:799-801.
38. Livak K J, Schmittgen T D. 2001. Analysis of relative gene expression data using real-time quantitative PCR and the 2−ΔΔCT method. Methods 25:402-408.
39. Kluger R. 1987. Thiamin diphosphate: a mechanistic update on enzymic and nonenzymic catalysis of decarboxylation. Chem Rev 87:863-876.
40. Stephens P E, Darlison M G, Lewis H M, Guest J R. 1983. The pyruvate dehydrogenase complex of Escherichia coli K12: nucleotide sequence encoding the dihydrolipoamide acetyltransferase component. Eur J Biochem 133:481-489.
41. Kale S, Arjunan P, Furey W, Jordan F. 2007. A dynamic loop at the active center of the Escherichia coli pyruvate dehydrogenase complex E1 component modulates substrate utilization and chemical communication with the E2 component. J Biol Chem 282:28106-28116.
42. Arjunan P, Nemeria N, Brunskill A, Chandrasekhar K, Sax M, Yan Y, Jordan F, Guest J R, Furey W. 2002. Structure of the pyruvate dehydrogenase multienzyme complex E1 component from Escherichia coli at 1.85 Å resolution. Biochemistry 41:5213-5221.
43. Nemeria N, Yan Y, Zhang Z, Brown A M, Arjunan P, Furey W, Guest J R, Jordan F. 2001. Inhibition of the Escherichia coli Pyruvate Dehydrogenase Complex E1 Subunit and Its Tyrosine 177 Variants by Thiamin 2-Thiazolone and Thiamin 2-Thiothiazolone Diphosphates. Evidence for reversible tight-binding inhibition. J Biol Chem 276:45969-45978.
44. Niersbach M, Kreuzaler F, Geerse R, Postma P, Hirsch H. 1992. Cloning and nucleotide sequence of the Escherichia coli K-12 ppsA gene, encoding PEP synthase. Mol Gen Genet 231:332-336.
45. Brice C, Kornberg H L. 1967. Location of a gene specifying phosphopyruvate synthase activity on the genome of Escherichia coli, K12. Proc R Soc Lond B Biol Sci 168:281-292.
46. Zhu Y, Eiteman M A, Altman R, Altman E. 2008. High glycolytic flux improves pyruvate production by a metabolically engineered Escherichia coli strain. Appl Environ Microbiol 74:6649-6655.
47. Causey T, Shanmugam K, Yomano L, Ingram L. 2004. Engineering Escherichia coli for efficient conversion of glucose to pyruvate. Proc Natl Acad Sci USA 101:2235-2240.
48. Davidi D, Noor E, Liebermeister W, Bar-Even A, Flamholz A, Tummler K, Barenholz U, Goldenfeld M, Shlomi T, Milo R. 2016. Global characterization of in vivo enzyme catalytic rates and their correspondence to in vitro kcat measurements. Proc Natl Acad Sci USA 113:3401-3406.
49. Garcia-Contreras R, Vos P, Westerhoff H V, Boogerd F C. 2012. Why in vivo may not equal in vitro-new effectors revealed by measurement of enzymatic activities under the same in vivo-like assay conditions. FEBS J 279:4145-4159.
50. Atsumi S, Li Z, Liao J C. 2009. Acetolactate synthase from Bacillus subtilis serves as a 2-ketoisovalerate decarboxylase for isobutanol biosynthesis in Escherichia coli. Appl Environ Microbiol 75:6306-6311.
51. Rahman M, Hasan M R, Oba T, Shimizu K. 2006. Effect of rpoS gene knockout on the metabolism of Escherichia coli during exponential growth phase and early stationary phase based on gene expressions, enzyme activities and intracellular metabolite concentrations. Biotechnol Bioeng 94:585-595.
52. Vemuri G, Altman E, Sangurdekar D, Khodursky A B, Eiteman M. 2006. Overflow metabolism in Escherichia coli during steady-state growth: transcriptional regulation and effect of the redox ratio. Appl Environ Microbiol 72:3653-3661.
53. Yang Y-T, Bennett G N, San K-Y. 2001. The effects of feed and intracellular pyruvate levels on the redistribution of metabolic fluxes in Escherichia coli. Metab Eng 3:115-123.

### Example 2

Innovative metabolic engineering tools and strategies are essential for building well-optimized microorganisms to produce biochemicals efficiently. Generally, to enhance the formation of specific product, the pathway to that biochemical is targeted, for example, using optimized enzymes (Atsumi and Liao, 2008; Zhang et al., 2010; Marcheschi et al., 2012) or plasmid constructs (Floras et al., 1996; Koffas et al., 2003; Yakandawala et al., 2008). The central goal of these approaches is to modify the expression, regulation, or catalytic activity of enzymes to increase metabolic carbon flux through the pathway leading to the product. Unfortunately, any heterologous pathway invariably competes with native metabolism supporting cell growth, and a key branchpoint exists between first enzyme in the pathway leading to the product, and the native enzymes. For example, products derived from pyruvate such as 2,3-butanediol (Ji et al., 2011), acetoin (Xiao and Lu, 2014) and isobutanol (Lan and Liao, 2013), must directly compete with various native pathways which could include pyruvate dehydrogenase (Reed and Willms, 1966), pyruvate oxidase (Williams and Hager, 1966), pyruvate decarboxylase (Gounaris et al., 1975), pyruvate carboxylase (Utter and Keech, 1963), lactate dehydrogenase (Tarmy and Kaplan, 1968) and numerous others.

Many strategies exist to eliminate or reduce competing pathways. For enzymes which are not required for growth, gene knockouts have become routine (Causey et al., 2003; Dittrich et al., 2005). Recently, studies have sought to reduce the activity of native pathways by modifying the promoter region of that enzyme. For example, the native aceE promoter was replaced with a weaker promoter to decrease the expression of aceE and optimize L-valine production in Corynebacterium glutamicum (Buchholz et al., 2013). Similarly, CRISPR interference has been applied to decrease the expression of the pyruvate dehydrogenase complex (PDHc) by targeting the promoter regions of pdhR and aceE, resulting in pyruvate accumulation by Escherichia coli (Ziegler et al., 2022). Alternatively, the intrinsic activity of a competing enzyme itself may be altered by making substitutions in key residues on that protein, which reduce but do not eliminate flux through that required native pathway (Tovilla-Coutiño et al., 2020). Using this approach, protein variants of AceE, the E1 component of PDHc, led to the accumulation of pyruvate in E. coli (Moxley and Eiteman, 2021). The use of PDHc variants as a metabolic engineering tool could increase the production of pyruvate-derived biochemicals but has not been demonstrated.

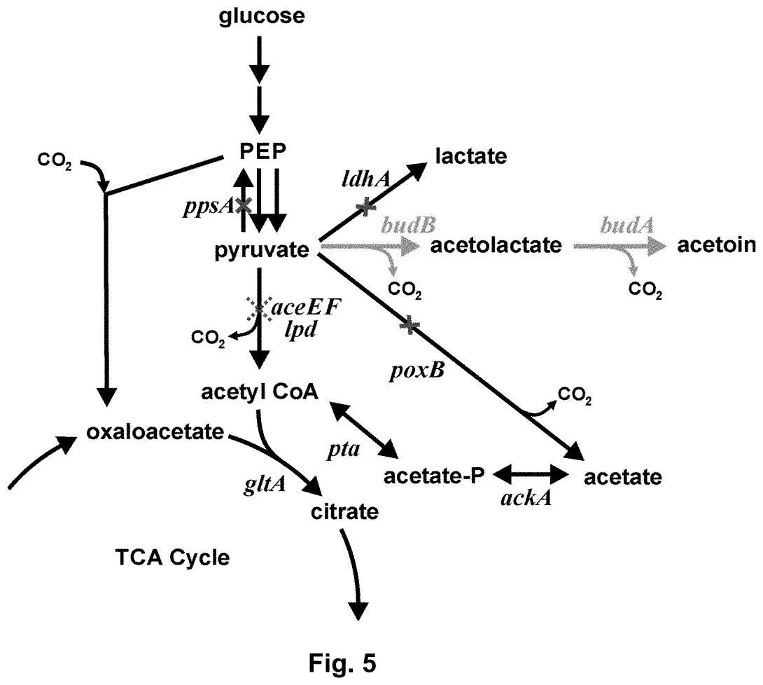

Acetoin, a metabolic precursor to 2,3-butanediol, is derived from pyruvate (FIG. 5) by the enzymes acetolactate synthase (ALS, Schloss et al., 1985) and acetolactate decarboxylase (ALDC, Løken et al., 1970). Acetoin is a volatile pale-yellow liquid with a yogurt odor and butter taste (Xiao and Lu, 2014). Commercially, acetoin is used as a flavor or fragrance in wide variety of products including foods, cigarettes, cosmetics, and biological pest controls (Xiao and Lu, 2014; Kandasamy et al., 2016). Acetoin is the simplest acyloin, a compound with a hydroxy group adjacent to a ketone, an important structure useful for the chemical synthesis of various products (Xiao and Lu, 2014). Industrially, acetoin is produced via chemical synthesis of fossil feedstocks, however, interest in microbially produced acetoin is growing, as consumer demand for natural products increases in the cosmetics and food industries (Xiao and Lu, 2014).

Many bacteria and yeast naturally produce acetoin as part of the butanediol fermentation pathway. E. coli lacks the complete butanediol fermentation pathway, but generates intermediate acetolactate during valine biosynthesis. One strategy for microbial acetoin production in native butanediol producers is to limit the conversion of acetoin to 2,3-butanediol. For example, overexpression of a water-forming NADH oxidase, encoded by nox in Serratia marcescens, a native 2,3-butanediol producer, achieved an acetoin titer of 75.2 g/L with a 52% reduction of 2,3-butanediol titer (Sun et al., 2012). This approach has been successfully applied to other native producers including as Bacillus subtilis and Klebsiella pneumoniae to increase acetoin production (Ji et al., 2013; Zhang et al., 2014). However, many native acetoin and 2,3-butanediol producers do not have GRAS (i.e., generally regarded as safe) status, leading to efforts to industrial strains such as E. coli to produce acetoin. The general strategy to engineer acetoin producing E. coli strains is to express heterologous genes encoding ALS and ALDC from native acetoin or butanediol producing microorganisms. For example, E. coli expressing alsS (encoding ALS) from B. subtilis and alsD (encoding ALDC) from Aeromonas hydrophila achieved an acetoin titer of 21 g/L (Oliver et al., 2013). Often paired with heterologous gene expression is the inactivation of by-product pathways. For example, knockouts in IdhA, pta, ackA, butA, and nagA decreased by-product formation and increased acetoin titer five-fold to 14 g/L in engineered C. glutamicum strains (Mao et al., 2017).

In E. coli and many other bacteria, pyruvate is primarily metabolized to acetyl-CoA by the PDHc under aerobic conditions. Thus, ALS directly competes for the substrate pyruvate with AceE, the pyruvate-binding subunit of the PDHc. Strategies aimed at reducing this competition include deleting aceE to direct pyruvate flux toward ALS. For example, 0.87 g/L of acetoin was produced when genes for ALS and ALDC were expressed in an E. coli strain containing a deletion in aceEF (Nielsen et al., 2010). While a deletion of aceE can increase the availability of pyruvate for acetoin production, under aerobic conditions this strategy requires the supplementation of an additional carbon source such as acetate (Langley and Guest, 1978; Tomar et al., 2003). Rather than eliminate the activity of AceE, an alternative strategy would be to reduce the activity of AceE through the use of enzyme variants. This strategy does not preclude other approaches, such as knockouts in genes coding for competing pathways not required for growth.

The aim of this study was to examine the use of AceE variants as a tool to increase the production of the pyruvate-derived biochemical acetoin. We hypothesized that reducing the flux through the PDHc would increase acetoin production during growth on defined medium with glucose as the sole carbon source. Alleles encoding AceE variants were engineered into E. coli W containing deletions in pyruvate oxidase (poxB), lactate dehydrogenase (IdhA), and phosphoenolpyruvate synthase (ppsA). An acetoin pathway containing ALS (budB) and ALDC (budA) from Enterobacter cloacae ssp. dissolvens was expressed in each strain to test the impact of AceE variants on acetoin yield.

Materials and Methods

Cultures were routinely grown on Lysogeny Broth during plasmid and strain construction, while aceE mutants were grown on TYA medium containing (per L) 10 g tryptone, 5 g NaCl, 1 g yeast extract, and 1 g sodium acetate trihydrate (Zhu et al., 2008). As needed, antibiotics were included in medium (final concentration): ampicillin (100 μg/mL), kanamycin (40 μg/mL), and chloramphenicol (20 μg/mL). For counter-selection against sacB, the medium was supplemented with 100 g/L sucrose, and NaCl was excluded.

The defined basal medium to which carbon/energy sources were added contained (per L): 8 g NH₄Cl, 1.2 g KH₂PO₄, 1.0 K₂HPO₄.3H₂O, 2.0 g K₂SO₄, 0.6 g MgSO₄.7H₂O, 0.25 mg ZnSO₄.7H₂O, 0.125 mg CuCl₂.2H₂O, 1.25 mg MnSO₄—H₂O, 0.875 mg CoCl₂.6H₂O, 0.06 mg H₃BO₃, 0.25 mg Na₂MoO₄.2H₂O, 5.5 mg FeSO₄.7H₂O, 20 mg Na₂EDTA.2H₂O, 20 mg citric acid, 20 mg thiamine HCl. In shake flask cultures, 20.9 g 3-[N-morpholino]propanesulfonic acid (100 mM MOPS) was used, while for batch processes, 25 mM MOPS was used. Thiamine was filtered sterilized, and other medium components were autoclaved in compatible mixtures, combined and then adjusted to a pH of 7.1 with 20% (w/v) NaOH.

Strains and Genetic Modifications

Strains used in this study are shown in Table 1. Gene knockouts in E. coli W were constructed by methods previously described (Datsenko and Wanner, 2000). Knockouts were selected on plates supplemented with kanamycin. Forward primers external to the target gene and reverse primers within the kanamycin resistance cassette were used to confirm proper chromosomal integration (Table S1). The kan^{R} marker was removed by expression of FLP recombinase from pCP20 (Datsenko and Wanner, 2000). Gene knockouts and removal of the markers were verified by PCR. To construct MEC1320, the chloramphenicol-sacB (cam-sacB) cassette and 200 bp of homology flanking aceE was amplified from purified genomic DNA of MEC813 (Moxley and Eiteman, 2021) and integrated into the aceE locus of MEC1319 expressing the lambda red system from pKD46. Nucleotide sequences of homologous regions used to integrate DNA into the aceE locus were identical in E. coli C (ATCC 8739) and E. coli W (ATCC 9637).

To construct MEC1329 and MEC1330, error-prone PCR was used to generate an aceE fragment with random mutations and was subsequently integrated into the aceE locus of MEC1122 expressing the lambda red system from pKD46. The error-prone PCR fragment of aceE was generated using Stratagene GeneMorph II Random Mutagenesis Kit (Stratagene California, San Diego, Calif., USA) using linearized (Spe1) pCM02 as template (Moxley and Eiteman, 2021). PCR was performed according to the manufacturer's specifications. The fragment was gel purified, and 100 ng used to transform MEC1122. Cells were recovered with TYA for 2 h at 30° C. The cells were centrifuged (5,000×g for 1 min) and washed with 1 mL 0.9% NaCl, then 100 ml of the washed recovery was plated to defined medium agar supplemented with 2.5 g/L glucose and 100 g/L sucrose. Plates were incubated at 30° C. for 2-3 days. Positive transformants were verified by PCR and subsequently screened for growth rate and pyruvate accumulation (data not shown). Two strains with decreased growth rates were chosen for sequencing to determine mutations.

Each aceE variant allele was PCR amplified from genomic DNA containing the respective allele and integrated into MEC1320 expressing the lambda red system from pKD46. Counter-selection against sacB was used to select mutants that lost the cam-sacB cassette by plating transformants on medium containing sucrose (Thomason, 2014). Colonies were confirmed by colony PCR, and point-mutated aceE genes were amplified from the chromosome, gel purified, and sequenced to confirm mutations.

Plasmid Construction

Plasmids used in this study are listed in Table S2. Plasmids were constructed using NEBuilder HiFi Assembly (New England Biolabs, Ipswich, Mass., USA). Phusion High-Fidelity Polymerase (New England Biolabs, Ipswich, Mass., USA) or PrimeStar Max High-Fidelity Polymerase (Takara Bio, Mountain View, Calif., USA) was used to amplify DNA for cloning and genome integration. Quick-DNA Miniprep and Zyppy Plasmid Miniprep Kits were used to purify genomic and plasmid DNA (Zymo Research, Irvine, Calif., USA). DNA Clean and Concentrator and Zymoclean Gel DNA Recovery Kits were used to purify PCR fragments (Zymo Research, Irvine, Calif., USA). Restriction enzymes were purchased from New England Biolabs. Plasmids were confirmed by restriction digest and sequencing (ACGT, Inc., Wheeling, Ill., USA).

To construct 44_ediss from 445_ediss gifted by Stefan Pflügl (Erian et al., 2018), primers were used to amplify a linear fragment containing the plasmid backbone, budA, and budB, then subsequently circularized to create the budB-budA operon. The primers were designed according to NEBuilder HiFi Assembly recommendations. The new plasmid 44_ediss was confirmed by restriction digest and sequencing.

Shake Flask Experiments

A single colony from an LB plate was used to inoculate 3 mL TYA. After 6-10 h of growth, this culture was used to inoculate 3 mL of basal medium with 5 g/L D-(+)-glucose to an initial optical density at 600 nm (OD) of 0.05. After 8-12 h of growth, this culture was used to inoculate three 500 mL baffled shake flasks containing 50 mL of basal medium with 5 g/L glucose to an OD of 0.02. All cultures were grown at 37° C. on a rotary shaker at 225 rpm. Flasks were sampled for measurement of growth rate and/or extracellular metabolite concentrations. Some flasks were supplemented with 2.34 g/L Na(CH₃COO)-3H₂O (1 g/L acetate) as described. Pyruvate yield for strains growing on glucose alone was calculated when the culture reached an OD of 2. Pyruvate yield for strains growing on glucose and acetate was calculated at the time acetate was depleted. Acetoin yield was calculated based on the end-point, corresponding to maximum acetoin titer.

Batch Processes

A single colony from an LB plate was used to inoculate 3 mL TYA. After 6-10 h, this culture was used to inoculate a 250 mL shake flask containing 50 mL of basal medium with 20 g/L glucose to an OD of 0.02. When the shake flask culture reached an OD of 1.5-2, the 50 mL were used to inoculate a 2.5 L bioreactor (Bioflo 2000, New Brunswick Scientific Co., New Brunswick, N.J., USA) containing 1.2 L basal medium with 40 g/L glucose. Duplicate batch processes were performed, and some cultures were supplemented with 18.72 g/L Na(CH₃COO).3H₂O (8 g/L acetate) as described.

Batch studies were conducted with a constant agitation of 400 rpm and at 37° C. Air and/or oxygen-supplemented air was sparged at 1.25 L/min to maintain a dissolved oxygen concentration above 40% of saturation. The pH was controlled at 7.0 using 30% (w/v) KOH/and 20% (w/v) H₂SO₄. Antifoam 204 (Sigma) was used as necessary to control foaming.

Analytical Methods

The optical density at 600 nm (OD) (UV-650 spectrophotometer, Beckman Instruments, San Jose, Calif., USA) was used to monitor cell growth. Samples were routinely frozen at −20° C. for further analysis, and thawed samples were centrifuged (4° C., 10000×g for 10 min), and filtered (0.45 μm nylon, Acrodisc, Pall Corporation, Port Washington, N.Y.). Liquid chromatography was used to quantify pyruvate, glucose and organic products using RI detection (Eiteman and Chastain, 1997).

Results

Variant Strain Screening for Pyruvate and Acetoin Yield

Pyruvate occupies a key node in central metabolism. Any biochemical product derived from pyruvate in an organism like E. coli must compete with native pathways including pyruvate dehydrogenase, lactate dehydrogenase and pyruvate oxidase. Although deletions in genes coding for lactate dehydrogenase, pyruvate oxidase and PEP synthase have minimal effect on aerobic cell growth and metabolism, a knockout in any of the three components of the pyruvate dehydrogenase complex (PDHc) results in a growth requirement for acetate (Tomar et al., 2003). An alternative approach to reducing flux through PDHc would be to reduce the intrinsic activity of the complex, for example, by amino acid substitutions affecting enzyme activity. Pyruvate accumulation at a yield of 0.66 g/g from glucose has previously been demonstrated in strains expressing variants of AceE, the E1 component of the PDHc (Moxley and Eiteman, 2021). The goal of this study was to examine the use of AceE variants for a product derived from pyruvate, acetoin. We compared five strains expressing different AceE variants for acetoin production. Our hypothesis was that the increased availability of pyruvate caused by a “bottleneck” through PDHc would increase the yield of pyruvate-derived acetoin.

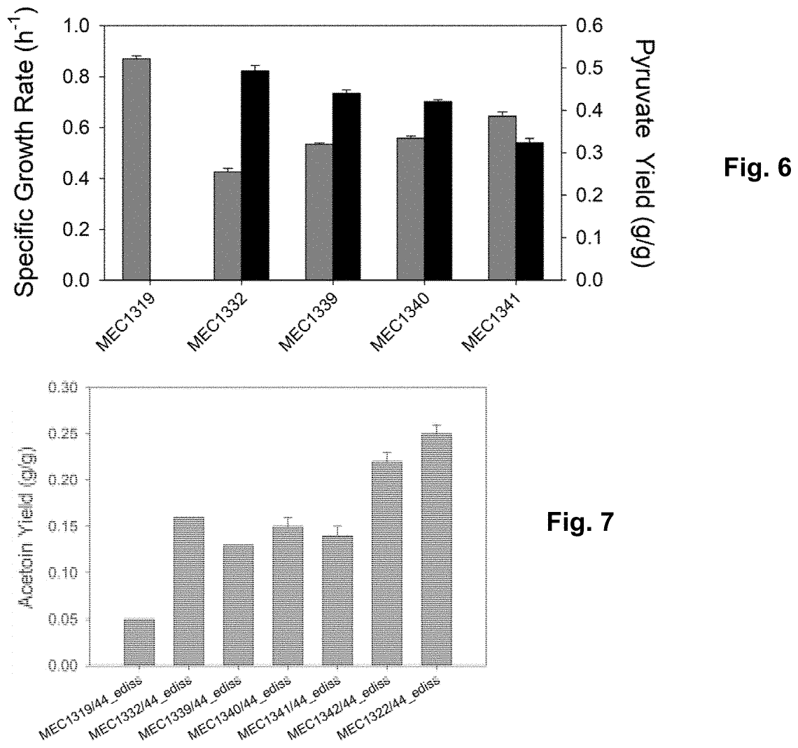

E. coli W containing deletions in the IdhA, poxB and ppsA genes (MEC1319) was chosen as the host for acetoin production. Several aceE variant alleles from E. coli C strains (Moxley and Eiteman, 2021) were introduced into MEC1319 and screened in triplicate shake flasks for growth rate and pyruvate yield (FIG. 6). We also examined MEC1322 containing a deletion of the aceE gene. MEC1319 attained a growth rate of 0.87±0.01 h⁻¹, and no pyruvate accumulated. Of the variants able to grow on glucose as the sole carbon source, MEC1332 (AceE[H106V] variant) attained the highest pyruvate yield of 0.49±0.01 g/g while MEC1341 (AceE[H106M]) attained the lowest pyruvate yield of 0.32±0.01 g/g. MEC1339 (AceE[N276S;R465C;V668A;Y696N]) and MEC1340 (AceE[V169A;P190Q;F532L] attained similar pyruvate yields of 0.44±0.01 g/g and 0.42±0.00 g/g, respectively. Pyruvate yield correlated inversely with maximum specific growth rate. MEC1332 attained the lowest growth rate of 0.43±0.01 h⁻¹, almost 50% lower than MEC1319 expressing the native AceE.

MEC1342 (AceE[H106M;E401A]) and MEC1322 (DaceE) exhibited limited growth on glucose as the sole carbon source (data not shown). Thus, for these two strains only, the medium was supplemented with 1 g/L acetate to support biomass formation. Upon acetate depletion, MEC1342 and MEC1322 converted glucose to pyruvate at yields of 0.39±0.02 g/g and 0.45±0.02 g/g, respectively.

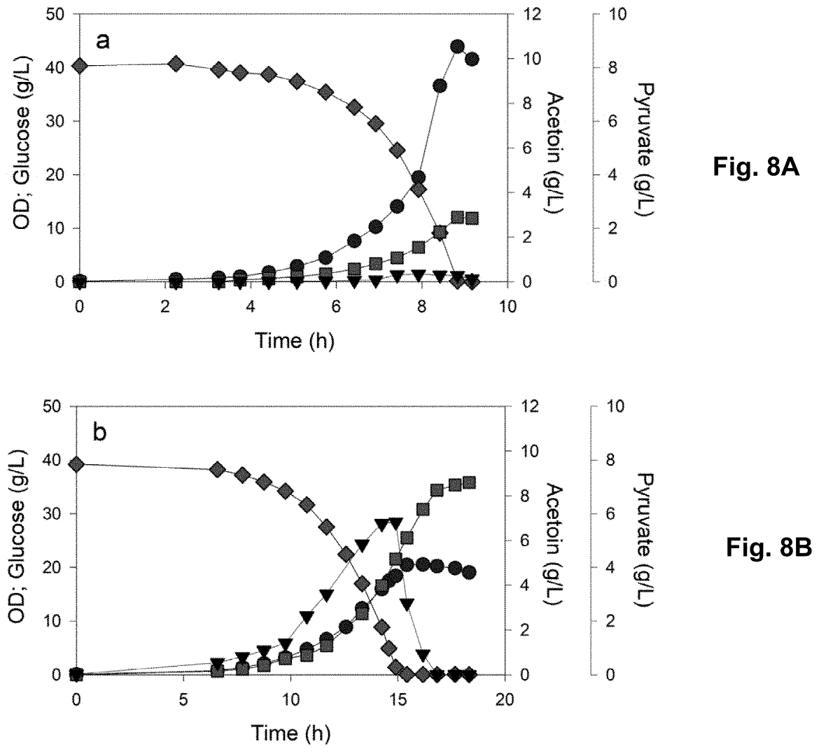

In order to understand the impact of each AceE variant on acetoin yield, an acetoin production pathway was introduced into each strain via transformation of the 44_ediss plasmid. The 44_ediss plasmid expresses acetolactate synthase, encoded by the budB gene, and acetolactate decarboxylase, encoded by the budA gene, each from E. cloacae ssp. dissolvens (FIG. 5). Each transformed strain was examined for acetoin generation in triplicate flask cultures containing 5 g/L glucose or 5 g/L glucose plus 1 g/L acetate (FIG. 7). Of the strains that grew on glucose as the sole carbon source, MEC1319/44_ediss displayed the lowest acetoin yield of 0.05±0.00 g/g while MEC1332/44_ediss attained the greatest yield of 0.16±0.00 g/g. MEC1341/44_ediss, MEC1340/44_ediss, and MEC1339/44_ediss attained yields of 0.13-0.15 g/g. For these strains, high pyruvate yield did not predict high acetoin yield. Strains with a severe restriction in PDHc and which therefore required acetate attained the highest acetoin yields. MEC1322/44_ediss and MEC1342/44_ediss attained yields of 0.25±0.01 g/g and 0.22±0.01 g/g, respectively.

Controlled Batch Processes

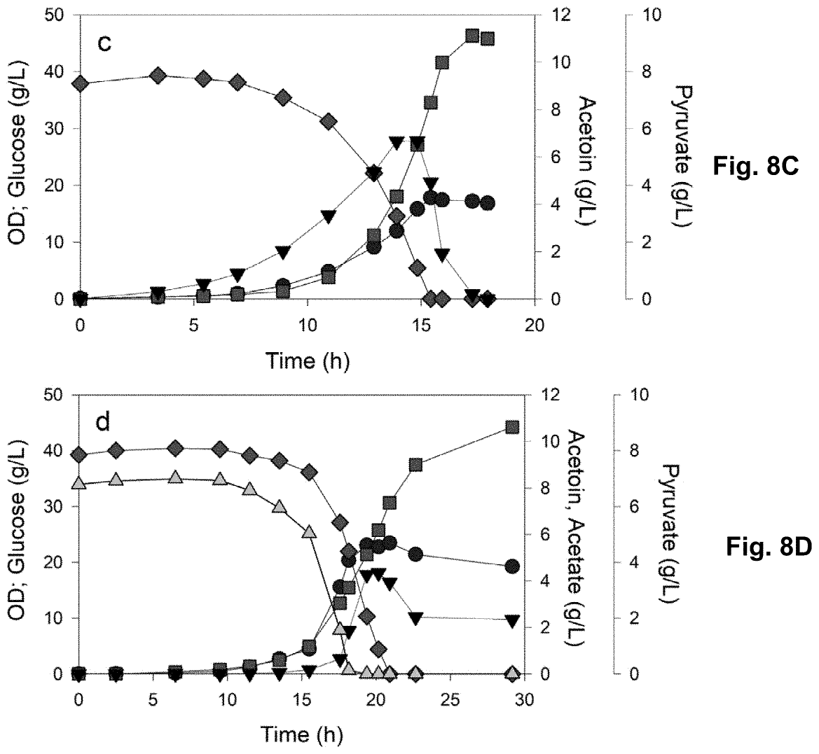

During the shake flask screening, acetoin titer was limited by low initial glucose concentration and potentially the lack of control of pH and dissolved oxygen. Thus, selected strains were grown in batch culture in controlled bioreactors using 40 g/L glucose (FIGS. 8A-8D). Medium was supplemented with 8 g/L acetate for the growth of MEC1342/44_ediss, which had limited ability to grow on glucose as the sole carbon source. MEC1319/44_ediss generated acetoin at a yield of 0.07±0.01 g/g and achieved a final titer of 2.66±0.01 g/L (FIG. 8A). MEC1340/44_ediss achieved a final acetoin titer of 8.49±0.00 g/L at a yield of 0.22±0.00 g/g (FIG. 8B) while MEC1332/42_ediss achieved a final acetoin titer of 11.15±0.00 g/L at a yield of 0.28±0.00 g/g (FIG. 8C). For both these two variants, the culture accumulated about 6 g/L pyruvate by the time that glucose as depleted, and then the pyruvate was itself depleted with continued generation of acetoin. MEC1342/44_ediss, grown on acetate-supplemented medium, converted glucose to acetoin at a yield of 0.26±0.01 g/g and achieved a final acetoin titer of 10.48±0.01 g/L (FIG. 8D). However, for this variant, after glucose and acetate were depleted, growth ceased, acetoin production slowed and nearly 2 g/L pyruvate remained.

Repeated Batch Process

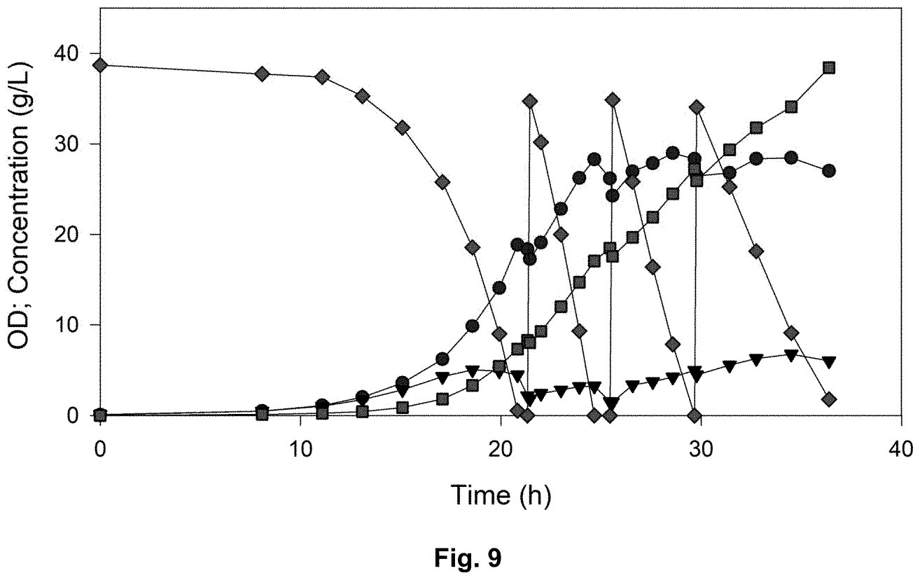

To maximize acetoin production, MEC1332/42_ediss was grown in batch culture and a 75 mL solution containing 44 g glucose and 40 mg kanamycin was added to the culture at the time of glucose depletion (FIG. 9). The glucose solution was added three times. MEC1332/42_ediss achieved an acetoin titer of 38.4 g/L with an overall yield of 0.27 g/g and productivity of 1.06 g/Lh.

### EXAMPLE 2 REFERENCES

Alper H, Fischer C, Nevoigt E, Stephanopoulos G. 2005. Tuning genetic control through promoter engineering. Proc Natl Acad Sci 102(36):12678-83.
Atsumi S, Liao J C. 2008. Directed evolution of Methanococcus jannaschii citramalate synthase for biosynthesis of 1-propanol and 1-butanol by Escherichia coli. App Environ Microbiol 74(24):7802-8.
Buchholz J, Schwentner A, Brunnenkan B, Gabris C, Grimm S, Gerstmeir R, Takors R, Eikmanns B J, Blombach B. 2013. Platform engineering of Corynebacterium glutamicum with reduced pyruvate dehydrogenase complex activity for improved production of L-lysine, L-valine, and 2-ketoisovalerate. Appl Environ Microbiol 79:5566-5575.
Causey T B, Zhou S, Shanmugam K T, Ingram L O. 2003. Engineering the metabolism of Escherichia coli W3110 for the conversion of sugar to redox-neutral and oxidized products: homoacetate production. Proc Natl Acad Sci 100(3):825-32.
Chang Y-Y, Cronan J E. 1982. Mapping nonselectable genes of Escherichia coli by using transposon Tn10: location of a gene affecting pyruvate oxidase. J Bacteriol 151:1279-1289.
Datsenko K A, Wanner B L. 2000. One-step inactivation of chromosomal genes in Escherichia coli K-12 using PCR products. Proc Natl Acad Sci USA 97:6640-6645.
Dittrich C R, Vadali R V, Bennett G N, San K Y. 2005. Redistribution of metabolic fluxes in the central aerobic metabolic pathway of E. coli mutant strains with deletion of the ackA-pta and poxB pathways for the synthesis of isoamyl acetate. Biotechnol Prog 21(2):627-31.
Eiteman M, Chastain M. 1997. Optimization of the ion-exchange analysis of organic acids from fermentation. Anal Chim Acta 338:69-75.
Erian A M, Gibish M, Pflügl S. 2018. Engineered E. coli W enables efficient 2,3-butanediol production from glucose and sugar beet molasses using defined minimal medium as economic basis. Microb Cell Fact 17, 190.
Floras N, Xiao J, Berry A, Bolivar F, Valle F. 1996. Pathway engineering for the production of aromatic compounds in Escherichia coli. Nat Biotechnol (5):620-3.
Gounaris A D, Turkenkopf I, Civerchia L L, Greenlie J. 1975. Pyruvate decarboxylase Ill: Specificity restrictions for thiamine pyrophosphate in the protein association step, sub-unit structure. Biochim Biophy Acta 405(2):492-9.
Ji X J, Huang H, Ouyang P K. 2011. Microbial 2, 3-butanediol production: a state-of-the-art review. Biotechnol Adv 29(3):351-64.
Ji X J, Xia Z F, Fu N H, Nie Z K, Shen M Q, Tian Q Q, Huang H. 2013. Cofactor engineering through heterologous expression of an NADH oxidase and its impact on metabolic flux redistribution in Klebsiella pneumoniae. Biotechnol Biofuels 6(1):1-9.
Kandasamy V, Liu J, Dantoft S H, Solem C, Jensen P R. 2016. Synthesis of (3R)-acetoin and 2,3-butanediol isomers by metabolically engineered Lactococcus lactis Sci Rep 6, 36769.
Koffas M A, Jung G Y, Stephanopoulos G. 2003. Engineering metabolism and product formation in Corynebacterium glutamicum by coordinated gene overexpression. Metabol Eng 5(1):32-41.
Lan E I, Liao J C. 2013. Microbial synthesis of n-butanol, isobutanol, and other higher alcohols from diverse resources. Biores Technol 135:339-49.
Langley D, Guest J. 1978. Biochemical Genetics of the α-Keto Acid Dehydrogenase Complexes of Escherichia coli K 12: Genetic Characterization and Regulatory Properties of Deletion Mutants. J Gen Microbiol 106:103-117.
Løken J P, Størmer F C. 1970. Acetolactate decarboxylase from Aerobacter aerogenes: purification and properties. Euro J Biochem 14(1):133-7.
Mao Y, Fu J, Tao R, Huang C, Wang Z, Tang Y J, Chen T, Zhao X. 2017. Systematic metabolic engineering of Corynebacterium glutamicum for the industrial-level production of optically pure D-(−)-acetoin. Green Chem 19(23):5691-702.
Marcheschi R J, Li H, Zhang K, Noey E L, Kim S, Chaubey A, Houk K N, Liao J C. 2012. A synthetic recursive “+1” pathway for carbon chain elongation. ACS Chem Biol 7(4):689-97.
Moxley W C, Eiteman M A. 2021. Pyruvate production by Escherichia coli by use of pyruvate dehydrogenase variants. Appl Environ Microbiol 87:e00487-21.
Nielsen D R, Yoon S H, Yuan C J, Prather K L J. 2010. Metabolic engineering of acetoin and meso-2,3-butanediol biosynthesis in E. coli. Biotechnol J 5, 274-284.
Oliver J W, Machado I M, Yoneda H, Atsumi S. 2013. Cyanobacterial conversion of carbon dioxide to 2, 3-butanediol. Proc Natl Acad Sci 110(4):1249-54.
Reed L J, Willms C R. 1955. Purification and resolution of the pyruvate dehydrogenase complex (Escherichia coli). Methods Enzymol 9:247-265.
Schloss J V, Van Dyk D E, Vasta J F, Kutny R M. 1985. Purification and properties of Salmonella typhimurium acetolactate synthase isozyme II from Escherichia coli HB101/pDU9. Biochemistry 24(18):4952-9.
Sun J A, Zhang L Y, Rao B, Shen Y L, Wei D Z. 2012. Enhanced acetoin production by Serratia marcescens H32 with expression of a water-forming NADH oxidase. Biores Technol 119:94-8.
Tarmy E M, Kaplan N O. 1968 Chemical characterization of D-lactate dehydrogenase from Escherichia coli B. J Biol Chem 243(10):2579-86.
Thomason L C, Sawitzke J A, Li X, Costantino N, Court D L. 2014. Recombineering: genetic engineering in bacteria using homologous recombination. Curr Protoc Mol Biol 106:1.16.1-1.16.39.
Tomar A, Eiteman, M A, Altman. 2003. The effect of acetate pathway mutations on the production of pyruvate in Escherichia coli. Appl Microbiol Biotechnol 62:76-82.
Tovilla-Coutiño D B, Momany C, Eiteman M A. 2020. Engineered citrate synthase alters acetate accumulation in Escherichia coli. Metabol Eng 61:171-180.
Utter M F, Keech D B. 1963. Pyruvate carboxylase. J Biol Chem 238(8):2603-8.
Williams F R, Hager L P. 1966. Crystalline flavin pyruvate oxidase from Escherichia coli: I. Isolation and properties of the flavoprotein. Arch Biochem Biophys 116:168-76.
Xiao Z, Lu J R. 2014. Strategies for enhancing fermentative production of acetoin: A review. Biotechnol Adv 32, 492-503.
Yakandawala N, Romeo T, Friesen A D, Madhyastha S. 2008. Metabolic engineering of Escherichia coli to enhance phenylalanine production. Appl Microbiol Biotechnol 78(2):283-91.
Zhang K, Li H, Cho K M, Liao J C. 2010. Expanding metabolism for total biosynthesis of the nonnatural amino acid L-homoalanine. Proc Natl Acad Sci 107(14):6234-9.
Zhang X, Zhang R, Bao T, Rao Z, Yang T, Xu M, Xu Z, Li H, Yang S. 2014. The rebalanced pathway significantly enhances acetoin production by disruption of acetoin reductase gene and moderate-expression of a new water-forming NADH oxidase in Bacillus subtilis. Metabol Eng 23:34-41.
Zhu Y, Eiteman M A, Altman R, Altman E. 2008. High glycolytic flux improves pyruvate production by a metabolically engineered Escherichia coli strain. Appl Environ Microbiol 74:6649-6655.
Ziegler M, Hagele L, Gebele T, Takors R. 2022. CRISPRi enables fast growth followed by stable aerobic pyruvate formation in Escherichia coli without auxotrophy. Eng Life Sci 22(2):70-84.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A modified bacterial strain comprising at least one mutation in a gene coding for proteins in a pyruvate dehydrogenase complex, wherein the mutation allows a cell to accumulate a product.

2. The modified bacterial strain of claim 1, wherein the modified bacterial strain is Escherichia coli.

3. The modified bacterial strain of claim 1, wherein the at least one mutation results in reduced activity of the pyruvate dehydrogenase complex and wherein the product is accumulated from glucose during aerobic growth.

4. The modified bacterial strain of claim 1, wherein the product is selected from pyruvate and products derived from pyruvate.

5. The modified bacterial strain of claim 1, wherein the product is acetoin.

6. The modified bacterial strain of claim 1, wherein the gene coding for proteins is an aceE gene and wherein the at least one mutation is selected from: [H106], [V169], [P190], [N276], [E401], [R465], [F532], [V668], [Y696], and a combination thereof.

7. The modified bacterial strain of claim 6, wherein the mutation is selected from aceE[E401A], aceE[H106V], and aceE[H106M].

8. The modified bacterial strain of claim 1, wherein the mutation is in an E1 component of the pyruvate dehydrogenase complex, an E2 component of the pyruvate dehydrogenase complex, or an E3 component of the pyruvate dehydrogenase complex.

9. The modified bacterial strain of claim 1, further comprising a deletion of phosphoenolpyruvate synthase (ppsA).

10. The modified bacterial strain of claim 1, further comprising a deletion of lactate dehydrogenase (IdhA) and pyruvate oxidase (poxB).

11. A modified bacterial strain comprising at least one mutation in a gene coding for proteins in a pyruvate dehydrogenase complex, wherein the at least one mutation allows a cell to accumulate pyruvate.

12. The modified bacterial strain of claim 11, wherein the at least one mutation results in reduced activity of the pyruvate dehydrogenase complex and wherein the pyruvate is accumulated from glucose during aerobic growth.

13. The modified bacterial strain of claim 11, wherein the strain yields about 0.50 g to 0.80 g of pyruvate per gram of glucose.

14. The modified bacterial strain of claim 11, wherein the strain generates at least 0.80 g/Lh of pyruvate.

15. The modified bacterial strain of claim 11, wherein the strain yields at least 0.65 g of pyruvate per gram of glucose.

16. The modified bacterial strain of claim 11, wherein the strain generates about 1.00 g/Lh to 2.00 g/Lh of pyruvate.

17. A modified bacterial strain comprising at least one mutation in a gene coding for proteins in a pyruvate dehydrogenase complex, wherein the at least one mutation allows a cell to accumulate acetoin.

18. The modified bacterial strain of claim 17, wherein the at least one mutation results in reduced activity of the pyruvate dehydrogenase complex and wherein the acetoin is accumulated from glucose during aerobic growth.

19. The modified bacterial strain of claim 17, wherein the strain yields about 0.20 g to 0.30 g of acetoin per gram of glucose.

20. The modified bacterial strain of claim 17, wherein the strain generates at least 0.80 g/Lh of acetoin.


SEQUENCE LISTING (57 sequences)

SEQ ID NO: 1 - DNA, 25 bp, Artificial Sequence
  Forward primer MEP166 for ldhA_F
ttaagcattcaatacgggtattgtg

SEQ ID NO: 2 - DNA, 25 bp, Artificial Sequence
  Forward primer MEP167 for ldhA_R
gtcattacttacacatcccgccatc

SEQ ID NO: 3 - DNA, 25 bp, Artificial Sequence
  forward primer MEP168 for aceE_F
tgagcgttctctgcgtcgtctggag

SEQ ID NO: 4 - DNA, 24 bp, Artificial Sequence
  forward primer MEP169 aceE_R
atcgccaacagagactttgatctc

SEQ ID NO: 5 - DNA, 18 bp, Artificial Sequence
  forward primer MEP288 poxB_F
ccggttgtcgctgcctgc

SEQ ID NO: 6 - DNA, 25 bp, Artificial Sequence
  forward primerMEP289 poxB_R
ttcaaacagatagttatgcgcggcc

SEQ ID NO: 7 - DNA, 24 bp, Artificial Sequence
  forward primer MEP290 ppsA_F
cgcacagaagcgtagaacgttatg

SEQ ID NO: 8 - DNA, 23 bp, Artificial Sequence
  forward primer MEP291 ppsA_R
cgtttaggtgaacgatcatgcgc

SEQ ID NO: 9 - DNA, 40 bp, Artificial Sequence
  forward primer MEP398 KSI-HA-ace-F
gacaaggcttctatggaagctgcaggaattcgatatcaag

SEQ ID NO: 10 - DNA, 39 bp, Artificial Sequence
  forward primer MEP399 KSI-HA-ace-R
gagtacggcgtttgattccgatccactagttctagagcg

SEQ ID NO: 11 - DNA, 38 bp, Artificial Sequence
  forward primer MEP400 ace-HA-KSI-F
gctctagaactagtggatcggaatcaaacgccgtactc

SEQ ID NO: 12 - DNA, 40 bp, Artificial Sequence
  forward primer MEP401 ace-HA-KSI-R
ttgatatcgaattcctgcagcttccatagaagccttgtcg

SEQ ID NO: 13 - DNA, 39 bp, Artificial Sequence
  forward primer mep402 cam-ha-ksi-
cgctctagaactagtggatctgtgacggaagatcacttc

SEQ ID NO: 14 - DNA, 40 bp, Artificial Sequence
  forward primer MEP403 cam-HA-KSI-R
cttgatatcgaattcctgcagatcaaagggaaaactgtcc

SEQ ID NO: 15 - DNA, 39 bp, Artificial Sequence
  forward primer MEP404 KSI-HA-cam-F
gacagttttccctttgatctgcaggaattcgatatcaag

SEQ ID NO: 16 - DNA, 39 bp, Artificial Sequence
  forward primer MEP405 KSI-HA-cam-R
gaagtgatcttccgtcacagatccactagttctagagcg

SEQ ID NO: 17 - DNA, 21 bp, Artificial Sequence
  forward primer MEP418 ace-seq-1
gaaatatctggaacaccgtgg

SEQ ID NO: 18 - DNA, 18 bp, Artificial Sequence
  forward primer MEP419 ace-seq-2
ccaaaggcaaagcgacag

SEQ ID NO: 19 - DNA, 27 bp, Artificial Sequence
  forward primer MEP420 ace-seq-3
cttactataaagaagacgagaaaggtc

SEQ ID NO: 20 - DNA, 40 bp, Artificial Sequence
  forward primer MEP421 aceE-HA-SCE-R
cacatagggataacagggtaatcatgggttattccttatc

SEQ ID NO: 21 - DNA, 40 bp, Artificial Sequence
  forward primer MEP422 aceE-HA-SCE-F
ggacagtagggataacagggtaataagtggttgctgacgc

SEQ ID NO: 22 - DNA, 40 bp, Artificial Sequence
  forward primer MEP423 cam-HA-SCE-R
ccacttattaccctgttatccctactgtccatatgcacag

SEQ ID NO: 23 - DNA, 40 bp, Artificial Sequence
  forward primer MEP424 cam-HA-SCE-F
cccatgattaccctgttatccctatgtgacggaagatcac

SEQ ID NO: 24 - DNA, 25 bp, Artificial Sequence
  forward primer MEP428 aceE-H106M-F
aactgggcggcatgatggcgtcctt

SEQ ID NO: 25 - DNA, 25 bp, Artificial Sequence
  forward primer MEP429 aceE-H106M-R
aaggacgccatcatgccgcccagtt

SEQ ID NO: 26 - DNA, 25 bp, Artificial Sequence
  forward primer MEP430 aceE-H106V-F
aactgggcggcgtgatggcgtcctt

SEQ ID NO: 27 - DNA, 25 bp, Artificial Sequence
  forward primer MEP431 aceE-H106V-R
aaggacgccatcacgccgcccagtt

SEQ ID NO: 28 - DNA, 25 bp, Artificial Sequence
  forward primer MEP432 aceE-H106N-F
aactgggcggcaacatggcgtcctt

SEQ ID NO: 29 - DNA, 25 bp, Artificial Sequence
  forward primer MEP433 aceE-H106N-R
aaggacgccatgttgccgcccagtt

SEQ ID NO: 30 - DNA, 40 bp, Artificial Sequence
  forward primer MEP434 aceE-E401A-F
ggcatgggcgacgcggctgcaggtaaaaacatcgcgcacc

SEQ ID NO: 31 - DNA, 40 bp, Artificial Sequence
  forward primer MEP435 aceE-E401A-R
ggtgcgcgatgtttttacctgcagccgcgtcgcccatgcc

SEQ ID NO: 32 - DNA, 40 bp, Artificial Sequence
  forward primer MEP440 aceE-E401D-F
ggcatgggcgacgcggctgatggtaaaaacatcgcgcacc

SEQ ID NO: 33 - DNA, 40 bp, Artificial Sequence
  forward primer MEP441 aceE-E401D-R
ggtgcgcgatgtttttaccatcagccgcgtcgcccatgcc

SEQ ID NO: 34 - DNA, 40 bp, Artificial Sequence
  forward primer MEP442 aceE-K403A-F
ggcatgggcgacgcggctgaaggtgcaaacatcgcgcacc

SEQ ID NO: 35 - DNA, 40 bp, Artificial Sequence
  forward primer MEP443 aceE-K403A-R
ggtgcgcgatgtttgcaccttcagccgcgtcgcccatgcc

SEQ ID NO: 36 - DNA, 40 bp, Artificial Sequence
  forward primer MEP444 aceE-K403N-F
ggcatgggcgacgcggctgaaggtaacaacatcgcgcacc

SEQ ID NO: 37 - DNA, 40 bp, Artificial Sequence
  forward primer MEP445 aceE-K403N-R
ggtgcgcgatgttgttaccttcagccgcgtcgcccatgcc

SEQ ID NO: 38 - DNA, 40 bp, Artificial Sequence
  forward primer MEP446 aceE-K403Q-F
ggcatgggcgacgcggctgaaggtcagaacatcgcgcacc

SEQ ID NO: 39 - DNA, 40 bp, Artificial Sequence
  forward primer MEP447 aceE-K403Q-R
ggtgcgcgatgttctgaccttcagccgcgtcgcccatgcc

SEQ ID NO: 40 - DNA, 35 bp, Artificial Sequence
  forward primer MEP448 aceE-K410N-F
catcgcgcaccaggttaacaaaatgaacatggacg

SEQ ID NO: 41 - DNA, 35 bp, Artificial Sequence
  forward primer MEP449 aceE-K410N-R
cgtccatgttcattttgttaacctggtgcgcgatg

SEQ ID NO: 42 - DNA, 35 bp, Artificial Sequence
  forward primer MEP450 aceE-K410Q-F
catcgcgcaccaggttcagaaaatgaacatggacg

SEQ ID NO: 43 - DNA, 35 bp, Artificial Sequence
  forward primer MEP451 aceE-K410Q-R
cgtccatgttcattttctgaacctggtgcgcgatg

SEQ ID NO: 44 - DNA, 35 bp, Artificial Sequence
  forward primer MEP452 aceE-K410M-F
catcgcgcaccaggttatgaaaatgaacatggacg

SEQ ID NO: 45 - DNA, 35 bp, Artificial Sequence
  forward primer MEP453 aceE-K410M-R
cgtccatgttcattttcataacctggtgcgcgatg

SEQ ID NO: 46 - DNA, 40 bp, Artificial Sequence
  forward primer MEP454 aceE-G395A-F
gctcataccattaaaggttacgcgatgggcgacgcggctg

SEQ ID NO: 47 - DNA, 40 bp, Artificial Sequence
  forward primer MEP455 aceE-G395A-R
cagccgcgtcgcccatcgcgtaacctttaatggtatgagc

SEQ ID NO: 48 - DNA, 39 bp, Artificial Sequence
  forward primer MEP602 aceE-H640V-F
aacggcgaaggtctgcaggtagaagatggtcacagccac

SEQ ID NO: 49 - DNA, 39 bp, Artificial Sequence
  forward primer MEP603 aceE-H640V-R
gtggctgtgaccatcttctacctgcagaccttcgccgtt

SEQ ID NO: 50 - DNA, 24 bp, Artificial Sequence
  forward primer MEP604 aceE-H142V-F
tacttccagggcgtaatctccccg

SEQ ID NO: 51 - DNA, 24 bp, Artificial Sequence
  forward primer MEP605 aceE-H142V-R
cggggagattacgccctggaagta

SEQ ID NO: 52 - DNA, 39 bp, Artificial Sequence
  forward primer MEP655 aceE-Y177F-F
gcaatggcctctcttccttcccgcacccgaaactgatgc

SEQ ID NO: 53 - DNA, 39 bp, Artificial Sequence
  forward primer MEP656 aceE-Y177F-R
gcatcagtttcgggtgcgggaaggaagagaggccattgc

SEQ ID NO: 54 - DNA, 20 bp, Artificial Sequence
  forward primer MEP852 aceE-RT-F
gtattggcgatctgtgctgg

SEQ ID NO: 55 - DNA, 20 bp, Artificial Sequence
  forward primer MEP853 aceE-RT-R
ctgtgaccatcttcgtgctg

SEQ ID NO: 56 - DNA, 20 bp, Artificial Sequence
  forward primer MEP856 rpoD-RT-F
tgatgctggctgaaaacacc

SEQ ID NO: 57 - DNA, 20 bp, Artificial Sequence
  forward primer MEP857 rpoD-RT-R
agttcaacggtgcccatttc